(19) United States
(12) Patent Application Publication
(10) Pub. No.: US 2021/0307349 A1
(43) Pub. Date: Oct. 7, 2021

(54) FORMULATIONS OF PHOSPHOLIPID COMPRISING OMEGA FATTY ACIDS

(71) Applicants: Sam L. Nguyen, Saratoga, CA (US); Volker Berl, Paramus, NJ (US)

(72) Inventors: Sam L. Nguyen, Saratoga, CA (US); Volker Berl, Paramus, NJ (US)

(73) Assignee: MyCell Technologies, LLC, Paramus, NJ (US)

(21) Appl. No.: 14/117,367

(22) PCT Filed: May 11, 2012
(86) PCT No.: PCT/US12/37623

§ 371 Date: Nov. 12, 2013

Related U.S. Application Data
(60) Provisional application No. 61/485,522, filed on May 12, 2011.

(51) Int. Cl. A23D 9/013; A23D 7/01; A23D 7/005; A23D 9/05; A23L 2/68; A23L 29/10; A23L 29/00; A23L 33/12; A61K 9/14; A61K 31/685; A61K 47/18; A61K 47/02

(52) U.S. Cl. CPC A23D 9/013; A23D 7/011; A23D 7/0053; A23D 9/05; A23L 2/68; A23L 29/10; A61K 47/02; A23L 29/055; A23L 29/015; A23L 33/12; A61K 9/14; A61K 31/685; A61K 47/183; A23L 29/045

(57) ABSTRACT

Disclosed herein are stabilized powder and aqueous formulations comprising a phospholipid comprising omega fatty acid and a micelle-forming surfactant. In one embodiment, the formulation further comprises a water soluble reducing agent, and/or a metal chelator, and/or a metal bisulfite reducing agent, or combinations thereof, wherein the formulation remains substantially clear and stable when stored at or below room temperature for a period of at least 6 months or at least 12 months; and methods for preparing these formulations.

### CROSS-REFERENCE TO RELATED APPLICATIONS

This application claims priority to U.S. Provisional Patent Application Ser. No. 61/485,522, filed May 12, 2011, the content of which is incorporated by reference for all purposes.

### SUMMARY OF THE PRESENT APPLICATION

A need exists for novel methods of preparing stabilized formulations comprising food, beverage, pharmaceutical or nutraceutical products containing phospholipid comprising omega fatty acids, such as krill oil. The following embodiments, aspects and variations thereof are exemplary and illustrative are not intended to be limiting in scope.

In one embodiment, there is provided a stable, water soluble formulation comprising: a) a phospholipid comprising omega fatty acid; and b) a solubilizing agent comprising the Formula (I), as defined herein.

In another embodiment, the water-soluble formulation further comprises a water soluble antioxidant. In another embodiment, the water-soluble formulation further comprises a metal chelator. In another embodiment, the water-soluble formulation further comprises a water-soluble reducing agent. In yet another embodiment, the water-soluble formulation further comprises a lipophilic antioxidant. In another embodiment, the water-soluble formulation further comprises a lipophilic reducing agent, or a combination of each of the above.

In one aspect of the above formulation, the omega fatty acid is selected from the group consisting of omega-3 fatty acids, omega-6 fatty acids, omega-9 fatty acids and omega-12 fatty acids. In another aspect, the omega fatty acid is selected from the group consisting of α-linolenic acid (ALA), stearidonic acid, eicosatetraenoic acid, eicosapentaenoic acid (EPA), docosapentaenoic acid, docosahexaenoic acid (DHA), linoleic acid, gamma-linolenic acid, eicosadienoic acid, dihomo-gamma-linolenic acid, arachidonic acid, docosadienoic acid, adrenic acid, docosapentaenoic acid, oleic acid, eicosenoic acid, mead acid, erucic acid and nervonic acid.

In another embodiment, there is provided a method for stabilizing a phospholipid comprising omega fatty acid (POFA) compound in an aqueous solution comprising contacting POFA with a composition comprising a micelle-forming surfactant, a water soluble reducing agent, and a metal chelator in water, at an elevated temperature, and for a sufficient period of time to dissolve the POFA. In another aspect, the micelle-forming surfactant is TPGS (polyoxyethanyl-a-tocopheryl succinate), Solutol HS 15 or Cremophor EL, or mixtures thereof. In a particular variation, the surfactant is TPGS-1000. In one variation, the metal chelator ethylenediaminetetraacetic acid. In another variation, the method further comprises contacting the aqueous solution with a metal bisulfite reducing agent.

In one embodiment, there is provided a stabilized aqueous formulation comprising a POFA, a micelle-forming surfactant, a water soluble reducing agent, a metal chelator and a reducing agent, wherein the formulation remains stable when stored at or below room temperature for a period of at least 6 months or at least 12 months. In one aspect, the POFA comprises an omega fatty acid. In another aspect, the omega fatty acid is an omega-3-fatty acid. In another aspect, the omega acid of the POFA further comprises an omega-3-, omega-6- and omega-9-fatty acid C₁-C₁₀ alkyl esters, C₁-C₅ alkyl esters, C₁-C₃ alkyl esters or C₂-C₅ alkyl esters; and mixtures thereof. In one aspect, the omega fatty acids is an omega-3-, omega-6- and omega-9-fatty acid ethyl ester. Accordingly, in another embodiment, there is provided a stabilized food, beverage, pharmaceutical or nutraceutical product comprising the aqueous formulation of the above.

In addition to the exemplary embodiments, aspects and variations described above, further embodiments, aspects and variations will become apparent by reference to the drawings and figures and by examination of the following descriptions.

### DETAILED DESCRIPTION OF THE PRESENT APPLICATION

### Definitions

Unless specifically noted otherwise herein, the definitions of the terms used are standard definitions used in the art of organic synthesis and pharmaceutical sciences. Exemplary embodiments, aspects and variations are illustratived in the figures and drawings, and it is intended that the embodiments, aspects and variations, and the figures and drawings disclosed herein are to be considered illustrative and not limiting.

### Definitions

The term “vitamin C derivative” as used herein means any compound that releases ascorbic acid (vitamin C) in vivo or in vitro, as well as solvates, hydrates and salts thereof. The term also includes vitamin C analogs wherein one or more of the hydroxyl groups of vitamin C are substituted with another moiety and wherein the vitamin C analog essentially retains the stabilizing activity of vitamin C in vitro or in vivo.

As used herein, the term “phospholipid” or “phospholipids” is recognized in the art, and refers to phosphatidyl glycerol, phosphatidyl inositol, phosphatidyl serine, phosphatidyl choline, phosphatidyl ethanolamine, as well as phosphatidic acids, ceramides, cerebrosides, sphingomyelins and cardiolipins.

As used herein, the term “solubilizing agent” is used interchangeably with the term “surfactant”. In one embodiment, the solubilizing agent is a nonionic, amphiphilic molecule, wherein the term amphiphilic means that the molecule includes at least one hydrophobic (e.g., lipid-soluble) moiety, such as a moiety derived from a tocopherol, a sterol, or a quinone (or derived hydroquinone, such as in the case of ubiquinone and ubiquinol) and at least one hydrophilic (e.g., water-soluble) moiety, such as polyethylene glycol or a simple sugar, carbohydrate or a carbohydrate derivative.

As used herein, the terms “stabilizer”, and “antioxidant”, are recognized in the art and refer to synthetic or natural substances that prevent or delay the oxidative or free radical or photo induced deterioration of a compound, and combinations thereof. Exemplary stabilizers include tocopherols, flavonoids, catechins, superoxide dismutase, lecithin, gamma oryzanol; vitamins, such as vitamins A, C (ascorbic acid) and E (tocopherol and tocopherol homologues and isomers, especially alpha and gamma- and delta-tocopherol) and beta-carotene (or related carrotenoids); natural components such as camosol, carnosic acid and rosmanol found in rosemary and hawthorn extract, proanthocyanidins such as those found in grape seed or pine bark extract, and green tea extract. In one variation, the vitamin E includes all 8-isomers (all-rac-alpha-tocopherol), and also include d,l-tocopherol or d,l-tocopherol acetate. In one variation, the vitamin E is the d,d,d-alpha form of vitamin E (also known as natural 2R,4R′,8R′-alpha-tocopherol). In another variation, the vitamin E includes natural, synthetic and semi-synthetic compositions and combinations thereof.

The term “reducing agent” is any compound capable of reducing a compound of the present application to its reduced form. “Reducing agent” includes lipophilic (e.g., lipid-soluble) reducing agents. In one example, the lipid-soluble reducing agent incorporates a hydrophobic moiety, such as a substituted or unsubstituted carbon chain (e.g., a carbon chain consisting of at least 10 carbon atoms). “Reducing agent” also includes hydrophilic (e.g., water-soluble) reducing agents. In one variation, the reducing agent that may be employed in the formulation is ubiquinol.

In one example, the reducing agent is a “water-soluble reducing agent” when the reducing agent dissolves in water (e.g., at ambient temperature) to produce a solution, as opposed to an otherwise inhomogeneous mixture, or even a two phase system. In one example, the reducing agent is a “water-soluble reducing agent” when it includes at least one (e.g., at least two) hydroxyl group(s) and does not include a large hydrophobic moiety (e.g., a substituted or unsubstituted linear carbon chain consisting of more than 10, 11, 12, 13, 14, 15, 16, 17, 18, 19 or 20 carbon atoms). In another example, the reducing agent is a “water-soluble reducing agent” when it includes at least one (e.g., at least two) hydroxyl group(s) and includes a substituted or unsubstituted linear carbon chain consisting of not more 6, 8, 10, 11, 12, 13, 14 or 15 carbon atoms. An exemplary water-soluble reducing agent is ascorbic acid. The term “water-soluble reducing agent” also includes mixtures of vitamin C with a lipophilic bioactive molecule of the present application. Water-soluble reducing agents can be derivatized to afford an essentially lipid-soluble reducing agent (pro-reducing agent). For example, the water-soluble reducing agent is derivatized with a fatty acid to give, e.g., a fatty acid ester. An exemplary lipid-soluble reducing agent is ascorbic acid-palmitate.

The term “water-soluble” when referring to a formulation or compositions of the present application, means that the formulation when added to an aqueous medium (e.g., water, original beverage) dissolves in the aqueous medium to produce a solution. In the absence of astaxanthin in the POFA that results in a reddish brown solution, the solution is essentially clear. In one example, the formulation dissolves in the aqueous medium without heating the resulting mixture above ambient temperature (e.g., 25° C.). The term “essentially clear” is defined herein.

The term “aqueous formulation” refers to a formulation of the present application including at least about 5% (w/w) water. In one example, an aqueous formulation includes at least about 10%, at least about 20%, at least about 30% at least about 40% or at least about 50% (w/w) of water.

The term “bioactive” refers to compounds and compositions of the present application. For example, a bioactive molecule is any compound having in vivo and/or in vitro biological activity. In one embodiment, the bioactive or bioactive molecule is a phospholipid comprising omega fatty acids (POFA), such as omega-fatty acids (or used interchangeably with omega fatty acid). Bioactive molecules or compositions also include those, which are suspected in the art to have biological activity (e.g., to have a positive effect on human health and/or nutrition). In one example, the biological activity is a desirable biological activity but can be accompanied by undesirable side-effects. Compounds with biological activity include pharmaceuticals, neutraceuticals and dietary supplements.

The terms “omega fatty acid(s)” and “omega-3-fatty acid(s)” of the phospholipids comprising krill oil are used interchangeably to mean the same composition, as known in the art, and include, for example, omega-3-, omega-6- and omega-9-fatty acids. Such omega-3 containing fatty acids present in the naturally occurring krill oil are the mono-phospholipid derivatives of omega fatty acids. In one aspect, hill oil contains the omega fatty acids EPA and DHA, in addition to the mono-phospholipid, although other combinations of omega-3 (or omega-6, or omega-9) fatty acids in place of either EPA or DHA, or both, are possible. Non-naturally occurring (or non-natural) omega fatty acids or omega-3-fatty acids include the non-phospholipid ester(s) of the omega-3-fatty acids. Such non-naturally occurring omega fatty acids include the ethyl esters of omega fatty acids that are, for example, the omega-3-, omega-6- and omega-9-fatty acids ethyl esters, and are also referred to as fatty acids ethyl esters (FAEE). In certain embodiments of the present application, the non-naturally occurring omega fatty acids used in the compositions of the present application comprise the C₁-C₁₀ alkyl esters, the C₁-C₅ alkyl esters, the C₁-C₃ alkyl esters or the C₂-C₅ alkyl esters. Further, in certain embodiments of the present application, the omega fatty acids used in the composition of the present application are phospholipids comprising fatty acids present in krill oil, or a mixture of the phospholipids of the omega fatty acids and (i.e., mixed with) the omega fatty acid esters, as defined herein. Accordingly, as used herein, unless otherwise noted, the term “phospholipid comprising omega fatty acids” as used in each aspects, variations and embodiments of the formulations of the present application include the natural (hill oil) phospholipids comprising omega fatty acids, the non-natural omega fatty acids, and their esters, and mixtures thereof, as defined herein. The krill oil may be purified to various different grades or quality as desired, depending on the desired characteristics of the formulation, resulting in a hill oil composition of different grades or quality (e.g., low grade, medium grade, high grade purity, with or without carotenoids, etc. . . . ) and different compositions, as disclosed herein.

Krill oil contains a phospholipid, in addition to esters of omega-3 fatty acids EPA and DHA. The composition of krill oil is significantly different from that of fish oil. The phospholipid(s) in krill oil include the choline-containing phospholipid classes, including phosphatidylcholine and lyso-phopsphatidylcholine. Thus, the naturally occurring levels of EPA and DHA in hill oil are necessarily lower than those found in fish oil. In contrast to fish oil that occurs naturally in the form of its triglycerides, krill oil is chemically distinct. Its phospholipid make up is regarded to offer greater passage through biomembranes that, likewise, are composed of phospholipids, leading to greater absorption and hence, bioavailability (95-99% for hill oil vs. 66% for fish oil, for example). Other constituents of hill oil that distinguish it from other sources of naturally occurring omega-3s (i.e., from fish or algae) include the presence of one or more antioxidants, such as vitamins E, D and A, as well as polyenic carotenoids such as astaxanthin and canthaxanthin. The former are water-insoluble, lipophilic vitamins, while the latter materials are also water-insoluble species known to provide protection against UV light and associated skin damage. The antioxidants in hill oil are reputed to afford stabilization to the omega-3s present, thus increasing shelf life. Relative to triglycerides containing three omega-3 PUFAs, it has been determined that the antioxidant potency of krill oil is 48 times greater than that of, e.g., fish oil on the ORAC scale.

The term “krill oil” or phospholipid comprising omega fatty acids (“POFA”) as used herein, means a natural or reconstituted (i.e., synthetic or unnatural) composition comprising phospholipids comprising omega fatty acids, such as omega-3 fatty acid, omega-6 fatty acid, omega-9 fatty acid, omega-12 fatty acid, and their mono-, diphospholipid isomers, and combinations thereof, and optionally, the POFA (or POFA composition or formulation) may further comprise omega fatty acids that is the omega fatty acid ethyl esters, such as the C₁-C₁₀ alkyl esters, the C₁-C₅ alkyl esters, the C₁-C₃ alkyl esters or the C₂-C₅ alkyl esters, and mixtures thereof. The POFA composition may comprise a monophosphate ester derivative (i.e., phospholipid esters, including the 1-, 2- or 3-isomer or mixtures thereof), a diphosphate derivative (including the 1,2- or 1,3-isomer or mixtures thereof), or a mixture of mono- and diphosphate derivatives and their isomers. In one aspect, the POFA composition may comprise omega-3 fatty acid, omega-6 fatty acid, omega-9 fatty acid, omega-12 fatty acid, and the non-phospholipid esters of the omega fatty acids, and mixtures thereof.

Given the variations in chemical composition of krill oil and its associated components, solubilization of naturally occurring material in water using nonionic surfactants such as TPGS as described in the present application, has not been established. That is, unlike omega-3s that have been solubilized both in their triglyceride (TG) and ethyl ester (EE) forms, phospholipid-derived omega-3s (i.e., phospholipid comprising fatty acids) as part of a totally different mixture, have not been previously shown to provide oxidatively stable, soluble compositions as described in the present application.

In certain embodiments, the ratios of surfactant to hill oil that are needed for solubilization purposes in water are significantly different than those used for either TGs or EEs. That is, in one aspect, the fundamental nature of the phospholipids themselves involves a polar, tetrahedral phosphorus atom rather than less polar carbons of the two remaining ester carbonyls. Without being bound by any particular theory advanced herein, it is believed that this difference is one aspect of the composition that alters the geometry of the molecule such that the attached omega-3s may self-associate into a pseudo-surfactant-like array. In the presence of a surfactant, these phospholipids may function as components of a mixed-component micelle, thereby imparting a synergistic effect that assists with solubilization in water and relying on less external surfactant. As disclosed herein, these properties result in a dramatic impact on the economics of providing hill oil-containing products where initial solubilization into an aqueous medium is especially beneficial. These novel properties are found to be especially useful for the production of enhanced waters that may contain useful levels of omega-3-rich krill oil that is stabilized by both its naturally occurring antioxidants, along with other additives. In one aspect, the composition of the present application provides formulations that have more extended product shelf life compared to fish oil.

In one aspect, the purification of naturally occurring krill oil leads to greatly enriched levels of highly bioavailable omega-3s. While the purification process would remove biologically insignificant oils, the antioxidants that, to some degree, protect the phospholipid esters of EPA and DHA may also be reduced. This implies that solubilization of these highly purified phospholipids in water may require additional antioxidants as stabilizing agents.

The term “pharmaceutical”, “pharmaceutical composition” or “pharmaceutical formulation” encompasses “neutraceutical” also referred to as “nutraceutical”), “neutraceutical composition” or “neutraceutical formulation”, respectively. Neutraceutical formulations or neutraceutical compositions may include a pharmaceutically acceptable carrier, such as those described herein.

The term “neutraceutical” or “nutraceutical” is a combination of the terms “nutritional” and “pharmaceutical”. It refers to a composition, which is known or suspected in the art to positively affect human nutrition and/or health.

The term “beverage” describes any water-based liquid, which is suitable for human consumption (i.e., food-grade). A typical beverage of the present application is any “original beverage” in combination with at least one bioactive lipophilic molecule of the present application. “Original beverage” can be any beverage (e.g., any marketed beverage). The term “original beverage” includes beers, carbonated and non-carbonated waters (e.g., table waters and mineral waters), flavored waters (e.g., fruit-flavored waters), mineralized waters, sports drinks (e.g., Gatorade®), smoothies, neutraceutical drinks, filtered or non-filtered fruit and vegetable juices (e.g., apple juice, orange juice, cranberry juice, pineapple juice, lemonades and combinations thereof) including those juices prepared from concentrates. Exemplary juices include fruit juices having 100% fruit juice (squeezed or made from concentrate), fruit drinks (e.g., 0-29% juice), nectars (e.g., 30-99% juice). The term “original beverage” also includes fruit flavored beverages, carbonated drinks, such as soft-drinks, fruit-flavored carbonates and mixers. Soft drinks include caffeinated soft drinks, such as coke (e.g., Pepsi Cola®, Coca Cola®) and any “diet” versions thereof (e.g., including non-sugar sweeteners). The term “original beverage” also includes teas (e.g., green and black teas, herbal teas) including instant teas, coffee, including instant coffee, chocolate-based drinks, malt-based drinks, milk, drinkable dairy products and beer. The term “original beverage” also includes any liquid or powdered concentrates used to make beverages.

The term “clear beverage” (e.g., clear juice) means any beverage clear (e.g., transparent) to the human eye. Typical clear beverages include carbonated or non-carbonated waters, soft drinks, such as Sprite®, Coke® or root beer, filtered juices and filtered beers. Typical non-clear beverages include orange juice with pulp and milk.

The term “non-alcoholic beverage” includes beverages containing essentially no alcohol. Exemplary non-alcoholic beverages include those listed above for the term “beverage”. The term “non-alcoholic beverage” includes beers, including those generally referred to as “non-alcoholic beers”. In one example, the non-alcoholic beverage includes less than about 10% alcohol by volume. In another example, the non-alcoholic beverage includes less than about 9% or less than about 8% alcohol by volume. In yet another example, the non-alcoholic beverage includes less than about 7%, less than about 6% or less than about 5% alcohol by volume.

The term “essentially stable to chemical degradation” refers to a bioactive molecule of the present application as contained in a formulation (e.g., aqueous formulation), beverage or other composition of the present application. In one example, “essentially stable to chemical degradation” means that the molecule is stable in its original (e.g., reduced) form and is not converted to another species (e.g., oxidized species; any other species including more or less atoms; any other species having an essentially different molecular structure), for example, through oxidation, cleavage, rearrangement, polymerization and the like, including those processes induced by light (e.g., radical mechanisms). Examples of chemical degradation include oxidation and/or cleavage of double bonds in unsaturated fatty acids and light-induced rearrangements of unsaturated molecules. Certain degradation products of omega-3-fatty acids include aldehydes. The molecule is considered to be essentially stable when the concentration of its original (e.g., reduced) form in the composition (e.g., aqueous formulation) is not significantly diminished over time. For example, the molecule is essentially stable when the concentration of the original form of the molecule remains at least 80% when compared with the concentration of the original form of the molecule at about the time when the composition is prepared. In another example, the molecule is essentially stable when the concentration of the original form remains at least about 85%, at least about 90% or at least about 95% of the original concentration. For example, an aqueous composition containing POFA at a concentration of about 50 mg/ml is considered essentially stable for at least 90 days when, at the end of the 90 days, the concentration of POFA in the aqueous composition remains at least about 40 mg/ml (80% of 50 mg/ml).

The term “essentially clear” is used herein to describe the compositions (e.g., formulations) of the present application in the absence of astaxanthin. The presence of astaxanthin in an aqueous solution turns the solution a reddish brown color. For example, the term “essentially clear” is used to describe an aqueous formulation or a beverage of the present application. In another example, the solubilizing agent (e.g., TWEEN-85, Solutol HS 15, Cremophor EL, TPGS or TPGS-1000) is present in a concentration that is above the critical micelle concentration (CMC) (i.e., the concentration that allows for spontaneous formation of micelles in water). For example, a typical CMC for TPGS in water is about 0.1 to about 0.5 mg/ml.

Alternatively, clarity, haziness or cloudiness of a composition of the present application can be determined by measuring the turbidity of the sample. This is especially useful when the composition is a beverage (e.g., water, soft-drink etc.). In one example, turbidity is measured in FTU (Formazin Turbidity Units) or FNU (Formazin Nephelometric Units). In one example, turbidity is measured using a nephelometer. Nephelometric measurements are based on the light-scattering properties of particles. The units of turbidity from a calibrated nephelometer are called Nephelometric Turbidity Units (NTU). In one example, reference standards with known turbidity are used to measure the turbidity of a sample.

The term “emulsion” as used herein refers to a lipophilic molecule of the present application emulsified (solubilized) in an aqueous medium using a solubilizing agent of the present application. In one example, the emulsion includes micelles formed between the lipophilic molecule(s) and the solubilizing agent. A typical aqueous medium, which is used in the emulsions of the present application, is water, which may optionally contain other solubilized molecules, such as salts, coloring agents, flavoring agents and the like. In one example, the aqueous medium of the emulsion does not include an alcoholic solvent, such as ethanol or methanol.

The term “micelle” is used herein according to its art-recognized meaning and includes all forms of micelles, including, for example, spherical micelles, cylindrical micelles, worm-like micelles and sheet-like micelles, and vesicles, formed in water, or mostly water.

The term “flavonoid” as used herein is recognized in the art. The term “flavonoid” includes those plant pigments found in many foods that are thought to help protect the body from disease (e.g., cancer). These include, for example, epi-gallo catechin gallate (EGCG), epi-gallo catechin (EGC) and epi-catechin (EC).

The term “tocopherol” includes all tocopherols, including alpha-, beta-, gamma- and delta tocopherol. The term “tocopherol” also includes tocotrienols.

Where substituent groups are specified by their conventional chemical formulae, written from left to right, they equally encompass the chemically identical substituents, which would result from writing the structure from right to left, e.g., —CH₂O— is intended to also recite —OCH₂—.

The term “metal chelator” or “metal chelating moiety” as used herein refers to a compound that combines with a metal ion, such as iron, to form a chelate structure. The chelating agents form coordinate covalent bonds with a metal ion to form the chelates. Accordingly, chelates are coordination compounds in which a central metal atom is bonded to two or more other atoms in at least one other molecule (ligand) such that at least one heterocyclic ring is formed with the metal atom as part of each ring. For the purposes of the present application, the metal chelator has demonstrated affinity for iron. These ions may be free in solution or they may be sequestered by a metal ion-binding moiety. The term “metal ion” as used herein refers to any physiological, environmental and/or nutritionally relevant metal ion. Such metal ions include certain metal ions such as iron, but may also include lead, mercury and nickel. When EDTA (or disodium EDTA or calcium disodium EDTA) is used in the present application to chelate iron, the chelate forms a Fe³⁺ ethylene-diaminetetraacetic acid (EDTA) complex.

When a residue is defined as “O⁻”, then the formula is meant to optionally include an organic or inorganic cationic counterion. For example, the resulting salt form of the compound is pharmaceutically acceptable.

Certain compounds of the present application possess asymmetric carbon atoms (chiral centers) or double bonds; the racemates, diastereomers, geometric isomers and individual isomers are encompassed within the scope of the present application. The graphic representations of racemic, ambiscalemic and scalemic or enantiomerically pure compounds used herein are taken from Maehr, J. Chem. Ed. 1985, 62: 114-120. Solid and broken wedges are used to denote the absolute configuration of a stereocenter unless otherwise noted. When the compounds described herein contain olefinic double bonds or other centers of geometric asymmetry, and unless specified otherwise, it is intended that the compounds include both E and Z geometric isomers. Likewise, all tautomeric forms are included.

Compounds of the present application can exist in particular geometric or stereoisomeric forms. The present application contemplates all such compounds, including cis- and trans-isomers, (−)- and (+)-enantiomers, diastereomers, (D)-isomers, (L)-isomers, the racemic mixtures thereof. All such isomers, as well as mixtures thereof, are intended to be included in this present application.

“Substituted or unsubstituted” or “optionally substituted” means that a group such as, for example, alkyl, aryl, heterocyclyl, (C₁-C₈)cycloalkyl, heterocyclyl(C₁-C₈)alkyl, aryl(C₁-C₈)alkyl, heteroaryl, heteroaryl(C₁-C₈)alkyl, and the like, unless specifically noted otherwise, may be unsubstituted or, may substituted by 1, 2 or 3 substituents selected from the group such as halo, nitro, trifluoromethyl, trifluoromethoxy, methoxy, carboxy, —NH₂, —OH, —SH, —NHCH₃, —N(CH₃)₂, —SMe, cyano and the like.

In one embodiment, there is provided aqueous compositions including a lipophilic bioactive molecule and a solubilizing agent described herein. In a particular aspect, the lipophilic bioactive molecule is a phospholipid comprising omega fatty acids (POFA; e.g., omega-3-, omega-6- or omega-9-fatty acids). In another embodiment, the omega fatty acids are the non-natural omega fatty acids that are the omega-3-, omega-6- and omega-9-fatty acids ethyl esters. In yet another embodiment, the omega fatty acids is the C₁-C₁₀ alkyl esters, the C₁-C₅ alkyl esters, the C₁-C₃ alkyl esters or the C₂-C₅ alkyl esters. In yet another embodiment, the omega fatty acids is a mixture of the natural and the non-natural omega fatty acids.

In another embodiment, the weight to weight (w/w) ratio of POFA to the solubilizing agent (POFA:solubilizing agent) where present, that may be used in the compositions or formulation of the present application is about 1:1 or less than 1:1, including about 0.9:1, 0.8:1, 0.7:1, 0.6:1, 0.5:1, 0.3:1, 0.2:1 and about 0.1:1. In another embodiment, the ratio of POFA:solubilizing agent, where present, may be about 0.09:1, 0.05:1, 0.03:1, 0.01:1, 0.05:1, 0.03:1, 0.01:1 or less. In another embodiment, the ratio of POFA:solubilizing agent, where present, may be about 0.009:1, 0.005:1, 0.003:1, 0.001:1, 0.005:1, 0.003:1, 0.001:1 or less.

In one embodiment, the weight to weight (w/w) ratio of the natural omega fatty acids to the non-natural omega fatty acids used in the compositions of the present application is about 100:1, about 95:5, about 90:10, about 80:20, about 70:30, about 60:40, about 55:45, about 50:50, about 45:55, about 40:60, about 30:70, about 20:80, about 10:90, about 5:95 or about 1:100.

In another embodiment, the formulation comprises POFA that is obtained from natural sources. In another embodiment, the formulation comprises POFA that has been reconstituted or synthetically prepared (or non-natural) from omega fatty acids, either deriving from fish oil omega fatty acids, krill oil or a combination of fish oil and hill oil. In one aspect, the natural or reconstituted POFA is a monophosphate ester derivative (including the 1-, 2- or 3-isomer or mixtures thereof), a diphosphate derivative (including the 1,2-, 1,3-isomer or mixtures thereof), or a mixture of mono- and diphosphate derivatives and their isomers. In another embodiment, the POFA composition further comprises a triphosphate derivative. In another embodiment, the natural and/or reconstituted POFA composition mixtures further comprises omega fatty acids that is the omega fatty acid ethyl esters, such as the C₁-C₁₀ alkyl esters, the C₁-C₅ alkyl esters, the C₁-C₃ alkyl esters or the C₂-C₅ alkyl esters, and mixtures thereof. In another embodiment, the natural POFA is greater than 35% pure, greater than 45% pure, greater than 55% pure, greater than 65% pure, greater than 75% pure, greater than 85% pure, greater than 90% pure or greater than 95% pure. In another embodiment, the reconstituted POFA is greater than 55% pure, greater than 65% pure, greater than 75% pure, greater than 85% pure, greater than 90% pure or greater than 95% pure. In another embodiment, the natural or reconstituted POFA is greater than 98% pure. In another aspect, the POFA comprising the omega-3 has an DHA:EPA ratio of about 1:1, 1:2, 1:3 or 1:5.

In one embodiment, the composition comprising the lipophilic bioactive molecules of the present application further comprises a mixture of POFA and at least a second lipophilic bioactive molecule. In one aspect, the second lipophilic bioactive molecule is CoQ₁₀. In another embodiment, the weight to weight (w/w) ratio of CoQ₁₀ to the natural POFA or the weight to weight (w/w) ratio of CoQ₁₀ to the reconstituted POFA, used in the compositions of the present application is about 100:1, about 95:5, about 90:10, about 80:20, about 70:30, about 60:40, about 55:45, about 50:50, about 45:55, about 40:60, about 30:70, about 20:80, about 10:90, about 5:95 or about 1:100. In a particular variation of the above, the omega fatty acid ester is the ethyl ester. In another embodiment of the formulation, the POFA is the natural POFA in combination with the reconstituted POFA combined with another lipophilic molecule, as provided herein.

These formulations have several advantages. This formulation can enable a consumer to ingest the lipophilic bioactive molecule in a liquid form, for example, in a beverage, such as water. In another embodiment, the present application provides formulations (e.g., aqueous formulations) of lipophilic bioactive molecules (e.g., natural and non-natural POFA comprising omega-3-, omega-6- or omega-9-fatty acids, and their esters, as defined herein) that include a solubilizing agent described herein, as well as a water-soluble reducing agent (also referred to as a stabilizer). The POFA in these formulations (especially aqueous formulations) are stable with respect to chemical degradation (e.g., oxidation). In one example, the chemical stability of the POFA is a result of a synergistic effect between the nature of the solubilizing agent and the water-solubility of the reducing agent (stabilizer): The solubilizing agent is an amphiphilic, nonionic surfactant, which in aqueous solutions allows the lipophilic molecule to be emulsified in “nanomicelles.” When the POFA is solubilized in the form of the micelles, a water-soluble reducing agent is surprisingly effective in preventing chemical degradation of the POFA in an aqueous solution. For example, the addition of a water-soluble reducing agent diminishes or prevents the degradation of the POFA and extends its average lifetime in solution, for example by at least 5 times. Molecules that are vulnerable to oxidation in aqueous solutions include the POFA (e.g., containing omega-3-, omega-6- or omega-9-fatty acids; or DHA).

In another example, the water-soluble reducing agent itself can be a compound with potential health benefits (e.g., vitamin C and other vitamins). The combination of two beneficial ingredients (POFA and stabilizer) in a single composition provides greater convenience to a consumer. Another benefit is that the surfactant supplies a nutrient in water (e.g., vitamin E, CoQ10, etc.).

The present application also provides a method for making aqueous, water-soluble POFA (e.g., omega-3-, omega-6- or omega-9-fatty acids) formulation of the present application. An exemplary process includes contacting an emulsion of a POFA, such as a phospholipid comprising omega-3-fatty acids in an aqueous medium (e.g., water) with a water-soluble reducing agent (e.g., vitamin C or a water-soluble derivative of vitamin C) and a metal chelating agent, such as ethylenediamine tetraacetic acid (EDTA). In addition, the process includes contacting the POFA composition in an aqueous medium with a water-soluble reducing agent, a metal chelating agent, and an aldehyde sequestering (by direct addition), or reducing agent, such as sodium bisulfite.

In one example, the POFA emulsion is formed using a solubilizing agent of the present application. The water-soluble formulations of the present application may be used to prepare beverages having mixtures of omega fatty acids dissolved therein.

### Compositions:

The present application provides formulations of POFA compositions. In one embodiment, the POFA is natural krill oil, reconstituted krill oil, purified hill oil or high grade krill oil, and mixtures thereof. In a specific variation, the POFA comprises the natural and non-natural omega fatty acids including omega-3-, omega-6- or omega-9-fatty acids, and their esters, and mixtures thereof, as defined herein. In another embodiment, the POFA comprises a non-natural omega fatty acids that are the omega-3-, omega-6- and omega-9-fatty acids ethyl esters. In yet another embodiment, the POFA comprises omega fatty acid that is the C₁-C₁₀ alkyl esters, the C₁-C₅ alkyl esters, the C₁-C₃ alkyl esters or the C₂-C₅ alkyl esters. In yet another embodiment, the POFA comprises omega fatty acids that is a mixture of the natural and the non-natural omega fatty acids. In one embodiment, the POFA comprising the omega fatty acids has a high concentration of the ester, such as the omega fatty acid ethyl esters. In one aspect, the concentration of the ethyl esters is at least about 60%, at least about 70%, at least about 80%, at least about 90%, or at least about 95% of the POFA.

In yet another embodiment, the lipophilic bioactive molecule as provided herein further comprises a mixture of POFA and at least a second lipophilic bioactive molecule. In one aspect, the second lipophilic bioactive molecule is ubiquinone (e.g., CoQ₁₀).

In one embodiment, the formulations comprise at least (a) a POFA or mixtures of POFA, as disclosed above, (b) a solubilizing agent, and (c) a metal chelating agent. In another embodiment, the formulations comprise at least (a) a POFA or mixtures of POFA, as disclosed above, (b) a solubilizing agent, (c) a water-soluble reducing agent, (d) a metal chelating agent, and e) an agent reactive towards aldehydes, such as sodium bisulfite. In one embodiment, the formulations comprise at least (a) a POFA of the present application, (b) a solubilizing agent, and (c) a metal chelating agent. In another embodiment, the formulations comprise at least (a) a POFA of the present application, (b) a solubilizing agent, (c) a water-soluble reducing agent, (d) a metal chelating agent, and (e) an agent reactive towards aldehydes, such as sodium bisulfite. In one embodiment, the weight to weight (wt/wt) ratio of POFA to the reducing agent, the metal chelating agent, and/or the agent reactive towards aldehydes, where present, may be about 0.09:1, 0.05:1, 0.03:1, 0.01:1, 0.05:1, 0.03:1, 0.01:1 or less. In another embodiment, the ratio of POFA to these agents, where present, may be about 0.009:1, 0.005:1, 0.003:1, 0.001:1, 0.005:1, 0.003:1, 0.001:1 or less. In another embodiment, the ratio of POFA to these agents, where present, may be about 0.0009:1, 0.0005:1, 0.0003:1, 0.0001:1, 0.0005:1, 0.0003:1, 0.0001:1 or less.

The inventors have discovered that the POFA or mixtures of POFA, which may be prone to chemical degradation (e.g., oxidation) can be stabilized using a combination of stabilizing agents that work both inside the micellar array, and outside in the aqueous medium. Thus, this approach includes a water-soluble reducing agent, when the molecule is formulated using a solubilizing agent of the present application (any micelle-forming surfactant; e.g., TPGS). An exemplary water-soluble reducing agent is selected from ascorbic acid (vitamin C) and water-soluble derivatives of vitamin C. Vitamin C is a convenient reducing agent because it is widely available and suitable for human consumption. In addition, aldehydes that are generated as by-products of degradation and result in undesirable smell and/or taste, may be neutralized by the addition of a reagent that reacts with the aldehyde, such as a bisulfite.

It was determined that water-soluble species that react with aldehydic by-products of oxidation (e.g., bisulfite, forming bisulfite addition compounds) are very effective in tandem with lipid-soluble reducing agents with respect to their capabilities to chemically stabilize the POFA in aqueous solutions. The present application further provides methods of making the formulations. The formulations of the present application can be used in a variety of products, such as foods, beverages, cosmetics and skin-care products (topical application), dietary supplements (e.g., formulated in soft-gelatine capsules) and nutraceuticals. In one embodiment, the present application provides a beverage including a formulation of the present application.

The following abbreviations are used throughout the application: Ub50—omega-3-fatty acid-50; TPGS—polyoxyethanyl-a-tocopheryl succinate (e.g., TPGS-1000, TPGS-600). A number following one of the above abbreviations (e.g., TPGS-600) indicates an average molecular weight of the polyoxyethanyl or poly(ethylene glycol) (PEG) moiety of the compound. A number followed by the abbreviation “Me” (e.g., TPGS-1000Me) indicates a polyoxyethanyl moiety capped with a methyl group (methoxypolyoxyethanyl or mPEG). Formulations:

In one embodiment, the present application provides a water-soluble formulation including a POFA or mixtures of POFA and bioactive agents as disclosed herein (e.g., a combination of phospholipids comprising omega-3s, and CoQ10 or ubiquinol), one or more water-soluble reducing agents, and a solubilizing agent of the present application. An alternative embodiment includes the above ingredients, but may rely on more than one solubilizing agent within any given formulation; i.e., a combination of surfactants (e.g., TPGS, TPGS-1000 or TWEEN-85, in any ratio). In one aspect, the present application provides a water-soluble formulation comprising POFA, comprising natural omega fatty acids or non-natural omega fatty acids (e.g., omega-3-, omega-6- or omega-9-fatty acids and their esters, as defined herein), a water-soluble reducing agent and a solubilizing agent of the present application. In one example, the solubilizing agent has a structure according to Formula (I):

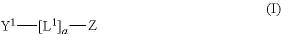

wherein:
a is 0 and 1;
L¹ is a linker moiety that covalently links the hydrophobic moiety Z and the hydrophilic moiety Y¹;
Y¹ is a linear or branched hydrophilic linker moiety comprising at least one polymeric moiety independently selected from poly(alkylene oxides) (e.g., PEG) and polyalcohols, and monoethers; and
Z is a hydrophobic moiety.

In another embodiment, there is provided a method for stabilizing a POFA composition comprising omega-3 fatty acid, omega-6 fatty acid, omega-9 fatty acid, omega-12 fatty acid, and the non-phospholipid esters of the omega fatty acids, and mixtures thereof, in an aqueous solution comprising contacting the POFA composition, with a composition comprising a micelle-forming surfactant for a sufficient period of time to dissolve the POFA.

In another embodiment, there is provided a stabilized aqueous formulation comprising a POFA selected from the group consisting of phospholipids comprising omega-3 fatty acid, omega-6 fatty acid, omega-9 fatty acid, omega-12 fatty acid, and their mono- and diphospholipid isomers, or further comprising the triphospholipid derivative, and combinations thereof, and a micelle-forming surfactant selected from the group consisting of TPGS (polyoxyethanyl-a-tocopheryl succinate), Solutol HS 15, Cremophor EL and combinations thereof, wherein the formulation remains substantially stable when stored at or below room temperature for a period of at least 6 months or at least 12 months.

In one embodiment, there is provided a stable, water soluble formulation comprising: a) a phospholipid comprising omega fatty acids; and b) one or more additives selected from the group consisting of a metal chelator, a water soluble reducing agent, a lipophilic reducing agent, a bisulfite salt, a metabisulfite salt or mixtures thereof. In one aspect, the formulation further comprises one or more solubilizing agent selected from the group consisting of solubilizing agents having a hydrophilic-lipophilic balance (HLB) of 8-18, HLB of 7-9 and HLB of 8-12, HLB of 13-15, or mixtures thereof. In another aspect of the formulation, the phospholipid comprising omega fatty acids is a natural or reconstituted phospholipid comprising omega fatty acids. In another aspect, the natural or reconstituted phospholipid comprising omega fatty acids comprise: a) omega fatty acids and their mono- and diphospholipid isomers, and combinations thereof; b) omega fatty acid ethyl esters selected from the group consisting of the C₁-C₁₀ alkyl esters, the C₁-C₅ alkyl esters, the C₁-C₃ alkyl esters or the C₂-C₅ alkyl esters, and mixtures thereof; c) a monophosphate ester derivative selected from the group consisting of 1-, 2- or 3-isomer or mixtures thereof; a diphosphate derivative selected from the group consisting of the 1,2- or 1,3-isomer or mixtures thereof; a triphosphate derivative; d) a mixture of mono-, di- and triphosphate derivatives and their isomers; or e) mixtures of a), b), c) and d) thereof; wherein the omega fatty acid is an omega-3 fatty acid, omega-6 fatty acid, omega-9 fatty acid, omega-12 fatty acid, and mixtures thereof. In one variation, the solubilizing agent comprises the Formula (I):

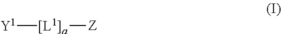

wherein: a is 0 and 1; L¹ is a linker moiety that covalently links the hydrophobic moiety Z and the hydrophilic moiety Y¹; Y¹ is a linear or branched hydrophilic moiety comprising at least one polymeric moiety independently selected from poly(alkylene oxides) and polyalcohols; and Z is a hydrophobic moiety. In another aspect of the above formulation, the phospholipids comprising omega fatty acids comprise a compound selected from the group consisting of omega-3 fatty acid, omega-6 fatty acid, omega-9 fatty acid, omega-12 fatty acid, the phospholipid esters of the omega fatty acids, the glyceride esters of the omega fatty acids, and the non-glyceride esters of the omega fatty acids, and mixtures thereof. In a variation of each of the above formulations, the phospholipid comprising omega fatty acids is selected from the group consisting of α-linolenic acid (ALA), stearidonic acid, eicosatetraenoic acid, eicosapentaenoic acid (EPA), docosapentaenoic acid, docosahexaenoic acid (DHA), linoleic acid, gamma-linolenic acid, eicosadienoic acid, dihomo-gamma-linolenic acid, arachidonic acid, docosadienoic acid, adrenic acid, docosapentaenoic acid, oleic acid, eicosenoic acid, mead acid, erucic acid and nervonic acid, and combinations thereof. In another variation, the solubilizing agent comprises the Formula (I), wherein: Z is selected from the group consisting of sterols, tocopherols, tocotrienol and omega fatty acids and derivatives or homologues thereof; L¹ is selected from a single bond, substituted or unsubstituted alkylene, substituted or unsubstituted heteroalkylene, substituted or unsubstituted arylene, substituted or unsubstituted heteroarylene and substituted or unsubstituted heterocycloalkylene; and Y¹ is a linear or branched hydrophilic moiety including at least one polymeric moiety, wherein each polymeric moiety is a member independently selected from poly(alkylene oxides) and polyalcohols. In another variation of the water soluble formulation, Y¹ is selected from the group consisting of poly(alkylene oxides) and monoethers therefrom, polyalcohols, polysaccharides, polyamino acids, polyphosphoric acids, polyamines and derivatives thereof; and L¹ is selected from the group consisting of a linear or branched C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄ or C₂₅-C₃₀ alkylene chain, optionally incorporating at least one functional group selected from the group consisting of ether, thioether, ester, carboxamide, sulfonamide, carbonate and urea groups. In one variation of each of the above formulations, the solubilizing agent is TPGS (polyoxyethanyl-a-tocopheryl succinate) or TPGS-1000 (D-alpha-tocopheryl polyethylene glycol 1000 succinate), wherein the tocopheryl is the natural tocopherol isomer or the un-natural tocopherol isomer. In another variation, the solubilizing agent is selected from the group consisting of Poloxamer 188, Polysorbate 80, Polysorbate 20, Vit E-TPGS, Solutol HS 15, PEG-40 Hydrogenated castor oil (Cremophor RH40), PEG-35 Castor oil (Cremophor EL), PEG-8-glyceryl capylate/caprate (Labrasol), PEG-32-glyceryl laurate (Gelucire 44/14), PEG-32-glyceryl palmitostearate (Gelucire 50/13); Polysorbate 85, Polyglyceryl-6-dioleate (Caprol MPGO), Mixtures of high and low HLB emulsifiers; Sorbitan monooleate (Span 80), Capmul MCM, Maisine 35-1, Glyceryl monooleate, Glyceryl monolinoleate, PEG-6-glyceryl oleate (Labrafil M 1944 CS), PEG-6-glyceryl linoleate (Labrafil M 2125 CS), Oleic acid, Linoleic acid, Propylene glycol monocaprylate (e.g. Capmul PG-8 or Capryol 90), Propylene glycol monolaurate (e.g., Capmul PG-12 or Lauroglycol 90), Polyglyceryl-3 dioleate (Plurol Oleique CC497), Polyglyceryl-3 diisostearate (Plurol Diisostearique) and Lecithin with and without bile salts, or combinations thereof. In another aspect of each of the above formulations, the water-soluble and lipophilic reducing agent are selected from the group consisting of L-ascorbic acid-6-palmitate, vitamin C and its salts alpha, beta, gamma, and delta tocopherol or mixtures of tocopherol, and alpha, beta, gamma and delta-tocotrienols or mixtures thereof. In another variation of the above formulations, the metal chelator is selected from the group consisting of ethylenediaminetetraacetic acid (EDTA), disodium EDTA and calcium disodium EDTA and mixtures thereof. In another variation of the above formulations, the bisulfite is sodium bisulfite, potassium bisulfite, sodium metabisulfite or potassium metabisulfite. In another variation of the above, the formulation, when dissolved in water, provides a solution that remains stable toward degradation when stored at or below room temperature for a period of at least 6 months. In yet another variation of the above, the ratio of the solubilizing agent to the phospholipid comprising fatty acid ranges from about to 2:1 to 0.01:1.

In another embodiment, there is provided a method for stabilizing a phospholipid comprising omega fatty acid comprising a compound selected from the group consisting of: a) omega fatty acids and their mono-, diphospholipid isomers, and combinations thereof; b) omega fatty acid ethyl esters selected from the group consisting of the C₁-C₁₀ alkyl esters, the C₁-C₅ alkyl esters, the C₁-C₃ alkyl esters or the C₂-C₅ alkyl esters, and mixtures thereof; c) a monophosphate ester derivative selected from the group consisting of 1-, 2- or 3-isomer or mixtures thereof; a diphosphate derivative selected from the group consisting of the 1,2- or 1,3-isomer or mixtures thereof; d) a mixture of mono- and diphosphate derivatives and their isomers; wherein the omega fatty acid is an omega-3 fatty acid, omega-6 fatty acid, omega-9 fatty acid, omega-12 fatty acid, and mixtures thereof; and mixtures of a), b), c) and d) thereof, in an aqueous solution; the method comprising contacting the phospholipid comprising omega fatty acid with a composition comprising one or more additives selected from the group consisting of a metal chelator, a water soluble reducing agent, a bisulfite salt, a metabisulfite salt or mixtures thereof, for a sufficient period of time to provide a stable formulation of the phospholipid comprising omega fatty acid. In one aspect of the method, there is further contacting the phospholipid comprising omega fatty acid with one or more solubilizing agent selected from the group consisting of solubilizing agents having a hydrophilic-lipophilic balance (HLB) of 8-18, HLB of 7-9 and HLB of 8-12, HLB of 13-15, or mixtures thereof, for a sufficient period of time to provide a stable formulation. In one aspect of the method, the solubilizing agent comprises the Formula (I):

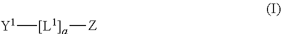

wherein: a is 0 and 1; L¹ is a linker moiety that covalently links the hydrophobic moiety Z and the hydrophilic moiety Y¹; Y¹ is a linear or branched hydrophilic moiety comprising at least one polymeric moiety independently selected from poly(alkylene oxides) and polyalcohols; and Z is a hydrophobic moiety. In one variation of the above methods, the solubilizing agent is Solutol HS 15, Cremophor EL, TPGS (polyoxyethanyl-a-tocopheryl succinate) or TPGS-1000 (D-alpha-tocopheryl polyethylene glycol 1000 succinate) or mixtures thereof, wherein the tocopheryl is the natural tocopherol isomer or the un-natural tocopherol isomer. In another aspect, the method provides contacting the phospholipid comprising omega fatty acid with the composition comprising the solubilizing agent for a sufficient period of time to dissolve the lipophilic bioactive compound is performed at an elevated temperature. In another aspect, the metal chelator is ethylenediaminetetraacetic acid (EDTA), disodium EDTA and calcium disodium EDTA or mixtures thereof.

In another embodiment, there is provided a stabilized aqueous emulsion of a phospholipid comprising fatty acid comprising: a) phospholipid comprising omega fatty acid; b) optionally, one or more solubilizing agent selected from the group consisting of solubilizing agents having a hydrophilic-lipophilic balance (HLB) of 8-18, HLB of 7-9 and HLB of 8-12, HLB of 13-15, or mixtures thereof; c) one or more additives selected from the group consisting of a metal chelator, a water soluble reducing agent, a lipophilic reducing agent, a bisulfite salt, a metabisulfite salt or mixtures thereof; d) a carrier or additive selected from the group consisting of HI-CAP 100 (National Starch), Emcap Starch, TICAMULSION FC (TIC GUMS), Spray gum F (gum acacia with Maltrin-100), natural vanillin, natural maltol, maltodextrin 10-DE and mixtures thereof; e) calcium disodium EDTA or disodium EDTA; f) sodium bisulfite, sodium metabisulfite, potassium bisulfite or potassium metabisulfite; and g) water, wherein the emulsion remains stable toward degradation when stored at or below room temperature for a period of at least 6 months. In one aspect, the solubilizing agent comprises the Formula (I):

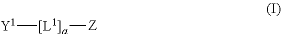

wherein: a is 0 and 1; L¹ is a linker moiety that covalently links the hydrophobic moiety Z and the hydrophilic moiety Y¹; Y¹ is a linear or branched hydrophilic moiety comprising at least one polymeric moiety independently selected from poly(alkylene oxides) and polyalcohols; and Z is a hydrophobic moiety. In another aspect of the above, the emulsion, when dissolved in water, the solution remains stable toward degradation when stored at or below room temperature for a period of at least 6 months.

In another embodiment, there is provided a stabilized powder composition of a phospholipid comprising omega fatty acid comprising: a) a phospholipid comprising omega fatty acid; b) TPGS (polyoxyethanyl-a-tocopheryl succinate), Solutol HS 15, Cremophor EL or mixtures thereof; c) a carrier or additive selected from the group consisting of HI-CAP 100 (National Starch), Emcap Starch, TICAMULSION FC (TIC GUMS), Spray gum F (gum acacia with Maltrin-100), natural vanillin, natural maltol, maltodextrin 10-DE and mixtures thereof; d) calcium disodium EDTA or disodium EDTA; and e) sodium bisulfite, potassium bisulfite, sodium metabisulfite or potassium metabisulfite; wherein the solution remains stable toward degradation when stored at or below room temperature for a period of at least 6 months. In another embodiment, there is provided a stabilized food, beverage, pharmaceutical or nutraceutical product comprising the above stabilized powder composition. In another embodiment, there is provided a method for preparing a dry powder composition comprising a stabilized phospholipid comprising omega fatty acid composition, the method comprising the steps of: (1) preparing an aqueous solution comprising a carrier or additive selected from the group consisting of HI-CAP 100 (National Starch), Emcap Starch, TICAMULSION FC (TIC GUMS), Spray gum F (gum acacia with Maltrin-100), natural vanillin, natural maltol, maltodextrin 10-DE and mixtures thereof; (2) combining the solution comprising the carrier or additive with a stabilized aqueous emulsion of a phospholipid comprising omega fatty acid comprising: a) a phospholipid comprising omega fatty acid; b) optionally, one or more solubilizing agent selected from the group consisting of solubilizing agents having a hydrophilic-lipophilic balance (HLB) of 8-18, HLB of 7-9 and HLB of 8-12, HLB of 13-15, and a solubilizing agent comprising the Formula (I):

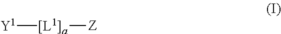

wherein: a is 0 and 1; L¹ is a linker moiety that covalently links the hydrophobic moiety Z and the hydrophilic moiety Y¹; Y¹ is a linear or branched hydrophilic moiety comprising at least one polymeric moiety independently selected from poly(alkylene oxides) and polyalcohols; and Z is a hydrophobic moiety; or mixtures thereof, and c) optionally, one or more additives selected from the group consisting of a metal chelator, a water soluble reducing agent, a lipophilic reducing agent, a bisulfite salt, a metabisulfite salt or mixtures thereof to form the pre-drying emulsion; and (3) drying the emulsion to form the dry powder composition comprising the stabilized phospholipid comprising omega fatty acid composition. In one variation of the method, the solubilizing agent is Solutol HS 15, Cremophor EL, TPGS (polyoxyethanyl-a-tocopheryl succinate) or TPGS-1000 (D-alpha-tocopheryl polyethylene glycol 1000 succinate) or mixtures thereof. In another variation, the drying step comprises of a spray drying of the emulsion to form the powder.

In one aspect, Z is selected from the group consisting of sterols (e.g., cholesterol or sitosterol), tocopherols (e.g., alpha-tocopherol), tocotrienol and omega fatty acids and derivatives or homologues thereof. In another aspect, the hydrophilic moiety is poly(ethylene glycol) (PEG) or methylated PEG (mPEG). The PEG moiety of the present application includes PEG-600 to PEG-2000. In one example, L¹ is selected from a single bond, substituted or unsubstituted alkylene, substituted or unsubstituted heteroalkylene, substituted or unsubstituted arylene, substituted or unsubstituted heteroarylene and substituted or unsubstituted heterocycloalkylene. In one embodiment, L¹ includes a linear or branched C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄ or C₂₅-C₃₀ alkylene chain, optionally incorporating at least one functional group. Exemplary functional groups according to this embodiment include ether, thioether, ester, carboxamide, sulfonamide, carbonate and urea groups. In a particular example, the solubilizing agent is selected from polyoxyethanyl-a-tocopheryl succinate (TPGS), TPGS-1000 (D-alpha-tocopheryl polyethylene glycol 1000 succinate) and combinations thereof. In one embodiment, the solubilizing agent is polyoxyethanyl-a-tocopheryl succinate (TPGS).

In one aspect, Y¹ is a linear or branched hydrophilic moiety including at least one polymeric moiety, wherein each polymeric moiety is a member independently selected from poly(alkylene oxides) (e.g., PEG) and polyalcohols. Exemplary lipophilic moieties are described herein, each of which is useful in this embodiment. In one example, the lipophilic moiety is poly(ethylene glycol) (PEG) or methylated PEG (mPEG). In one embodiment, Y¹ is selected from poly(alkylene oxides) (i.e., polyethers), polyalcohols, polysaccharides (e.g., polysialic acid), polyamino acids (e.g., polyglutamic acid, polylysine), polyphosphoric acids, polyamines and derivatives thereof. Exemplary poly(alkylene oxides) include polyethylene glycol (PEG) and polypropylene glycol (PPG). PEG derivatives include those, in which the terminal hydroxyl group is replaced with another moiety, such as an alkyl group (e.g., methyl, ethyl or propyl). In one example, the hydrophilic moiety is methyl-PEG (mPEG).

PEG is usually a mixture of oligomers characterized by an average molecular weight. In one example, the PEG has an average molecular weight from about 200 to about 5000. In another aspect, PEG has an average molecular weight from about 500 to about 1500. In another aspect, PEG has an average molecular weight from about 500 to about 800 or about 900 to about 1200. In one example, the PEG is PEG-600 or is PEG-750. Both linear and branched PEG moieties can be used as the hydrophilic moiety of the solubilizing agent in the practice of the invention. In one aspect, PEG has between 1000 and 5000 subunits. In one aspect, the PEG is PEG 1000. In another aspect, PEG has between 100 and 500 subunits. In yet another aspect, PEG has between 10 and 50 subunits. In one aspect, PEG has between 1 and 25 subunits. In another aspect, PEG has between 15 and 25 subunits. PEG has between 5 and 100 subunits. In another aspect, PEG has between 1 and 500 subunits.

In one aspect, the ratio of the natural and reconstituted POFA, optionally comprising omega-3-, omega-6- or omega-9-fatty acids and their esters, to the solubilizing agent is from about 1:0.1 (w/w), about 1:0.3, or a range of about 1:0.3 (w/w) to about 1:20 (w/w); or from about 1:1 (w/w) to about 1:20 (w/w), from about 1:1 (w/w) to about 1:10 (w/w); from about 1:1.3 (w/w) to about 1:5 (w/w), from about 1:2 (w/w) to about 1:4 (w/w), or is about 1:3 (w/w). In another variation, the ratio of the POFA to the solubilizing agent is from about 1:0.1 (w/w) to about 1:0.3 (w/w), about 1:0.3 (w/w) to about 1:1 (w/w), or from about 1:0.5 (w/w) to about 1:2 (w/w).

### Water-Soluble Reducing Agent:

Certain fatty acids, including the POFA of the present application, are known to be unstable toward oxidation, resulting in the formation of unstable hydroperoxides that break down to different volatile aldehydes that cause an undesirable odor and rancid taste. Microencapsulation using spray dry emulsions and complex coacervation technologies have been used to stabilize fatty acids for use in food products, but such methods do not provide stable aqueous formulations. C. J. Barrow et al, Lipid Technology, May 2007, Vol. 19, No. 5, 108-111. In one embodiment, the water-soluble reducing agent contained in the formulation (e.g., aqueous formulation) protects the POFA molecule from chemical degradation (e.g., oxidative and/or light-induced processes). For example, addition of vitamin C, a water-soluble vitamin C derivative, or a water-insoluble version of vitamin C to a formulation containing DHA/EPA and TPGS serve to prolong the chemical stability of POFA in the aqueous formulation for at least several weeks. In other embodiments, the water-soluble reducing agent (e.g. based on vitamin C) is added to the formulation in an amount sufficient to both reduce and stabilize the POFA molecule after reduction. For example, the POFA composition and a solution of a solubilizing agent in water (e.g., TPGS, TPGS-1000 or TWEEN-85) are mixed. Upon mixing of the components, micelles of a small particle size are formed (e.g., average particle size between about 10 and about 30 nm). A water-soluble reducing agent, such as vitamin C or a vitamin C derivative, is then added. Excess of water-soluble reducing agent serves to protect against omega-3-fatty acids degradation (e.g., oxidation). In this function, the water-soluble reducing agent can be considered a stabilizer. In one example, the reducing agent is added in an over-stoichiometric mole ratio with respect to the POFA composition, optionally comprising omega-3-fatty acids, such as omega-3-, omega-6- or omega-9-fatty acids and mixtures thereof. In another embodiment, the ratio of POFA to water-soluble reducing agent in the formulation is between about 100:1 and about 1:20 (w/w), or between about 50:1 and about 1:10 (w/w), or between about 20:1 and about 1:10 (w/w), or between about 10:1 and about 1:10 (w/w), or between about 1:1 (w/w) and about 1:10 (w/w), between about 1:1 and about 1:8 (w/w), about 1:1 and about 1:6 (w/w) or between about 1:1 and about 1:4 (w/w). In yet another embodiment, the ratio of POFA to water-soluble reducing agent in the formulation is between about 1:1 and about 1:3 (w/w), or between about 1:1 and about 1:2 (w/w). A person of skill in the art will understand that at least part of the reducing agent can be present in its “oxidized” form. For example, when vitamin C is used as the water-soluble reducing agent, at least part of the vitamin C can be present in the formulation as dehydroascorbic acid. In one example, the ratio of POFA to water-soluble reducing agent in the formulation is between about 100:1 and about 10:1 (w/w).

In one example according to any of the above embodiments, the POFA in the formulation are essentially stable to chemical degradation (e.g., oxidation). In one example, the formulation is essentially stable for at least 30, 60, 90, 120, 160, 180 days, or at least about 6 months, 9 months or about 12 months when stored at a temperature below about 25° C. (e.g., about 4° C. or about 10° C.). Typically, the formulations are stored at about 4° C. At this temperature, the formulations are typically stable for at least 4, 5, 6 or 12 months. In one example, according to any of the above embodiments the formulation is contained in a soft-gelatin capsule. A person of skill will understand that formulations suitable for incorporation into soft-gelatin capsules typically contain less than about 5%, less than about 4%, less than about 3% and less than about 2% (w/w) of water. Hence, in one example, the formulation includes less than 5% (w/w) of water.

The POFA compositions in the above formulations can be any lipophilic bioactive molecule or mixtures thereof. In one example, according to any of the above embodiments, the lipophilic bioactive molecule is selected from the phospholipid comprising omega fatty acids (POFA), wherein the omega fatty acids comprises docosahexaenoic acid (DHA), eicosapentaenoic acid (EPA) and alpha-linolenic acid (ALA), omega-6-fatty acid, omega-9-fatty acid, carotenoids, essential oils, flavor oils and lipophilic vitamins. Exemplary carotenoids include lutein, astaxanthin, lycopene, fucoxanthin and canthaxanthin.

In one example, according to any of the above embodiments, the formulation is an aqueous formulation and includes at least about 5% (w/w) of water, at least about 10%, at least about 20%, at least about 30%, at least about 40% or at least about 50% (w/w) of water. In another example, the aqueous formulation includes more than 50% (w/w) of water. For example, the aqueous formulation includes at least about 55%, at least about 60%, at least about 65%, at least about 70%, at least about 75% or at least about 80% (w/w) of water. In a further example, the aqueous formulation includes more than 80% (w/w) water. For example, the aqueous formulation includes at least about 85%, at least about 90%, at least about 92%, at least about 94% or at least about 96% (w/w) of water.

In one example, the POFA are solubilized in the aqueous formulation through the formation of micelles that are generated most commonly by the self-aggregation of surfactant molecules, or alternatively, by inclusion of the bioactive as part of the micellar array; i.e., mixed micelles formed between the POFA and the solubilizing agent. The particle size of the formed micelles in solution may be measured using a dynamic light scattering (DLS) detector.

In another example, the aqueous formulation does not include an alcoholic solvent, although such inclusion is possible when part of the solubilizing agent (e.g., as in Cremophore, which contains ethanol). Exemplary alcoholic solvents include solvents, such as ethanol, methanol, propanol, butanol and higher alcohols (e.g., C₅-C₂₀ alcohols). Alcoholic solvents also include polyhydric alcohols, such as ethylene glycol, propylene glycol, glycerol and the like. The term “alcoholic solvent” does not include polymers, such as polymeric versions of the above listed polyhydric alcohols (e.g., poly(alkylene oxides)), such as PEG or PPG).

In one example, according to any of the above embodiments, the concentration of POFA in the formulation is at least about 20 mg/mL and can be as high as about 60, about 80, about 100 or more than about 100 mg/mL. In one example, the concentration of POFA in the aqueous formulation of the present application is at least about 1 mg/mL, at least about 5 mg/mL, at least about 10 mg/mL, at least about 20 mg/mL, at least about 30 mg/mL, at least about 40 mg/mL, at least about 50 mg/mL, at least about 60 mg/mL, at least about 70 mg/mL or at least about 80 mg/mL, at least about 85 mg/mL, at least about 90 mg/mL, at least about 95 mg/mL or at least about 100 mg/mL, at least about 110 mg/mL, at least about 120 mg/mL, at least about 130 mg/mL, at least about 140 mg/mL, at least about 150 mg/mL, at least about 160 mg/mL, at least about 170 mg/mL, at least about 180 mg/mL, at least about 190 mg/mL or at least about 200 mg/mL. In another example, the concentration of POFA in the aqueous formulation is greater than 200 mg/mL.

In one embodiment, the present application provides a water-soluble formulation comprising bioactive agent or mixtures of bioactive agents, including the POFA as disclosed herein, a water-soluble reducing and/or antioxidizing agent, a solubilizing agent, a metal chelating agent, and a bisulfite salt or a metabisulfite salt. In another embodiment, the present application provides a water-soluble formulation comprising POFA composition, a water-soluble antioxidant and/or reducing agent, a solubilizing agent, a metal chelating agent, and a bisulfite salt or a metabisulfite salt. In one aspect, the chelating agent is EDTA and the bisulfite salt is sodium bisulfite. In one example, the solubilizing agent has a structure according to Formula (I) described herein.

In particular variations of each of the above aspects and embodiments, the formulation may comprise the natural POFA and reconstituted POFA and TPGS-1000; natural, non-natural and synthetic surfactants and mixtures of surfactants, including, for example, two or more surfactants of differing structural types (e.g., TPGS-1000 and Tween-80), two or more surfactants from within the same structural class (e.g., TPGS-1000+TPGS-600). In another variation of the above formulations, the formulations may also comprise any of the above combinations as their free alcohols, or as their ether or ester derivatives (of their PEG portion). In another particular variation of the above formulations, the formulations may also comprise antioxidants that are lipophilic in nature (e.g., vitamin C palmitate), hydrophilic in nature (e.g., vitamin C), and any combinations of these, including more than one of each in any formulations. In another particular variation of the above formulations, the formulations may also comprise chelating agents that are lipophilic in nature, hydrophilic in nature (e.g., EDTA, HEDTA, DTPA and NTA), and any combinations of these, and in any number (i.e., more than one of each in any formulation) or ratio. In another particular variation of the above formulations, the formulations may also comprise salts such as salts that are lipophilic in nature (e.g., ammonium salts, such as R₄N⁺X⁻), hydrophilic in nature (e.g., NaHSO₃), and any combinations of these, and in any number (i.e., more than one of each in any formulation) or ratio, that may vary with each application. According to the present formulations, variations of each of the above natural and non-natural omega fatty acids and their esters, the surfactants, the antioxidants, chelating agents, lipophilic and hydrophilic salts, and each of these elements and their combinations, may be used to provide the stable, water soluble bioactive agents such as the omega fatty acids formulations of the present application.

In one example according to any of the above embodiments, the POFA or mixtures of POFA formulation is essentially stable to chemical degradation. In one example, the POFA is essentially stable for at least 30, 60, 180 days, or at least 6 months, 9 months or 12 months, when stored at a temperature below about 25° C. (e.g., about 4° C. or about 10° C.). Typically, omega fatty acids formulations are stored at about 4° C. At this temperature, the POFA composition and formulations are stable for at least 90 days, at least 6 months or at least 12 months.

Another advantage of the above POFA formulations is that they can be light in color or reddish in color where astaxanthin is present. In another example, the POFA are emulsified in the formulation in the form of micelles that include the POFA and the solubilizing agent. In one example, the POFA concentration in the aqueous formulations of the present application is at least about 20 mg/mL and can be as high as about 60, about 80, about 100 or more than about 100 mg/mL.

### Beverages:

In another example, the present application provides a mixture between a formulation of the present application (e.g., a water-soluble formulation) and an original beverage to create a beverage of the present application. The original beverage can be any beverage (e.g., a clear beverage). Exemplary original beverages are described herein and include carbonated or non-carbonated waters, flavored waters, soft drinks and the like. In one example, the mixture (beverage of the present application) includes between about 1 mg/L and about 1000 mg/L of solubilized POFA. In another example, the mixture includes between about 10 mg/L and about 500 mg/L of solubilized POFA, between about 10 mg/L and about 450 mg/mL, between about 10 mg/L and about 400 mg/mL, between about 10 mg/L and about 350 mg/mL, between about 10 mg/L and about 300 mg/mL, or between about 10 mg/L and about 250 mg/mL of solubilized POFA. In a further example, the mixture includes between about 20 mg/L and about 250 mg/L, between about 20 mg/L and about 200 mg/mL, between about 20 mg/L and about 150 mg/mL, between about 20 mg/L and about 100 mg/mL, or between about 20 mg/L and about 80 mg/mL, between about 20 mg/L and about 60 mg/mL, between about 20 mg/L and about 40 mg/mL of solubilized POFA. According, in one aspect, the beverage may comprise of about 1,000 mg or less, 500 mg or less, and about 250 mg or less of solubilized POFA. In one aspect, the beverage may comprise of a range of about 10 mg to about 500 mg per serving. In another aspect, the beverage may comprise of a range of about 25 mg to about 500 mg per serving. In certain aspects, the beverage may have two servings. In certain variation of the beverage, the beverage may comprise about 15% to about 30% of the daily recommended value of the omega fatty acids in the POFA.

In one embodiment, the concentration of the POFA in the formulation provides the daily recommended dose for omega-3 fatty acids. In one aspect, the formulation provides up to about 500 mg of omega-3 fatty acids per serving.

In a particular example according to any of the above embodiments, the present application provides a mixture between the POFA formulation of the present application (e.g., an aqueous phospholipid comprising omega fatty acids formulation) and an original beverage (e.g., carbonated or non-carbonated water) to form a POFA beverage. In another aspect, the present application provides a non-alcoholic beverage comprising (a) solubilized POFA, (b) a water-soluble reducing agent of the present application (e.g., vitamin C), (c) a solubilizing agent, (d) a metal chelating agent, and (e) sodium bisulfite.

In another embodiment, the POFA beverage contains between about 1 mg/L and about 1000 mg/L of solubilized omega fatty acids, between about 10 mg/L and about 500 mg/L of solubilized POFA, between about 10 mg/L and about 450 mg/mL, between about 10 mg/L and about 400 mg/mL, between about 10 mg/L and about 350 mg/mL, between about 10 mg/L and about 300 mg/mL, or between about 10 mg/L and about 250 mg/mL of solubilized POFA. In a further example, the mixture includes between about 20 mg/L and about 250 mg/L, between about 20 mg/L and about 200 mg/mL, between about 20 mg/L and about 150 mg/mL, between about 20 mg/L and about 100 mg/mL, or between about 20 mg/L and about 80 mg/mL, between about 20 mg/L and about 60 mg/mL, between about 20 mg/L and about 40 mg/mL of solubilized POFA.

In a further example according to any of the above embodiments, the beverage further includes a coloring agent and/or a flavoring agent. It is possible to add one or more fruit and/or vegetable juice concentrates and/or flavor improvers to the beverage. For example, a mixture of about LIMETTE citrus (e.g., about 1.38 g/l), cassis (e.g., about 1.04 g/l), mango (e.g., about 1.04 g/l) or combinations thereof, can be added to the beverage. In another example, maltodextrin (e.g., about 20 g/l), fructose (e.g., about 50 g/l) or combinations thereof can be added to the beverage. In another example, the finished beverage is subjected to a primary and, optionally, a secondary filtration.

In yet another example according to any of the above embodiments, the POFA can be solubilized and stabilized in the beverage. For example, the beverage is essentially free of POFA precipitation.

In addition, the beverage can be enriched with vitamins. In one example, the beverage includes at least one B vitamin. Exemplary B-vitamins include vitamin B1, vitamin B2, vitamin B3, vitamin B5, vitamin B6 and vitamin B12. In another example, the beverage includes vitamin E. In one example, the vitamin is first formulated into an aqueous composition, which is subsequently added to the beverage. The solubilizing agent used to solubilize the vitamin can be the same solubilizing agent used to solubilize the POFA.

### Lipophilic Bioactive Molecule:

The bioactive molecule of the present application can be any lipophilic molecule. In one example, the lipophilic bioactive molecule is selected from compounds with a water-solubility that can be increased using a solubilizing agent of the present application. In another example, the bioactive lipophilic molecule is a molecule associated with pharmaceutical or neutraceutical value. The term “lipophilic bioactive molecule” includes derivatives of such molecules (e.g., esters or amides thereof) and combinations thereof. For example, the lipophilic bioactive molecule has at least one free OH or COOH group, which can be converted to an ester group. In another example, the lipophilic bioactive molecule has at least one free primary or secondary amino group, which can be converted to an amide or related derivatives (e.g, sulfonamides, carbamates, etc.).

### Oils, Fats and Fatty Acids:

The term essential oil also includes fragrances and flavoring oils (e.g., fruit flavor oils, citrus flavor, almond flavor). Exemplary oils derived from animals include animal fats, such as tallow (e.g., beef tallow), butter, chicken fat, lard, dairy butterfat, or combinations thereof. In another exemplary embodiment, the lipophilic bioactive molecule is selected from krill oil comprising at least one fatty acids (e.g., an essential fatty acid). In another exemplary embodiment, the lipophilic bioactive molecule is selected from krill oil comprising at least one type of an omega-3 fatty acids, an oil comprising at least one type of an omega-6 fatty acid, an oil comprising at least one type of an omega-9 fatty acid and an oil comprising at least one type of an omega-12 fatty acid. Exemplary types of omega-3 fatty acid, omega-6 fatty acid, omega-9 fatty acid and omega-12 fatty acid are disclosed herein. In another embodiment, the POFA comprises fatty acids selected from the group consisting of an omega-3 fatty acid, an omega-6 fatty acid, an omega-9 fatty acid, and an omega-12 fatty acid. In another embodiment, the lipophilic bioactive molecule is an essential fatty acid (EFA), such as a linolenic acid. In another exemplary embodiment, the POFA comprises an omega-3 unsaturated fatty acid, such as alpha-linolenic acid (ALA), docosahexaenoic acid (DHA), eicosapentaenoic acid (EPA), stearidonic acid, eicosatetraenoic acid and docosapentaenoic acid. In another exemplary embodiment, the POFA comprises an omega-6 unsaturated fatty acid, such as linoleic acid, gamma-linolenic acid and arachidonic acid. In yet another exemplary embodiment, the POFA comprises an omega-9 unsaturated fatty acid, such as oleic acid, eicosenoic acid and erucic acid, as well as conjugated linoleic acid (CLA). In a further exemplary embodiment, the POFA comprises an omega-12 unsaturated fatty acid. The term “fatty acid” also includes any derivative of those compounds, such as mixed phospholipids, triglycerides, diglyceride esters and alkyl esters, such as methyl- and ethyl esters; and combinations thereof. In one aspect, the POFA comprises the triglyceride esters. Additional fatty acids of the present application are summarized below.

Exemplary Omega-3, Omega-6 and Omega-9 Fatty Acids Common Name Lipid Name Chemical Name Omega-3 Fatty Acids: α-Linolenic acid (ALA), stearidonic acid; eicosatetraenoic acid; eicosapentaenoic acid (EPA), docosapentaenoic acid, docosahexaenoic acid (DHA); Omega-6 Fatty Acids: Linoleic acid, gamma-linolenic acid, eicosadienoic acid, dihomo-gamma-linolenic acid, arachidonic acid, docosadienoic acid, adrenic acid, docosapentaenoic acid; Omega-9 Fatty Acids: Oleic acid, eicosenoic acid, mead acid, erucic acid, nervonic acid.

In one example, the formulation includes from about 0.01% (w/w) to about 0.1% (w/w) of POFA, from about 0.01% (w/w) to about 0.5% (w/w), from about 0.01% (w/w) to about 1% (w/w), from about 0.05% (w/w) to about 0.25% (w/w), from about 0.1% (w/w) to about 1% (w/w), from about 0.1% (w/w) to about 0.75% (w/w), from about 1% (w/w) to about 3% (w/w), from about 1% (w/w) to about 10% (w/w), from about 1% (w/w) to about 20% (w/w), from about 1% (w/w) to about 30% (w/w), from about 1% (w/w) to about 40% (w/w), from about 5% to about 50% by weight, or from about 10% to about 30% (w/w), for example, from about 15% to about 25% (w/w).

### Solubilizing Agents Wherein Z is a Tocopherol or a Tocotrienol:

In another embodiment, Z is selected from a substituted or unsubstituted tocopherol and a substituted or unsubstituted tocotrienol. In one example, Z is an α-, β-, γ-, or δ-tocopherol. α-(+)-Tocopherol (natural) and α-(±)-tocopherol (synthetic) are preferred tocopherols, with synthetic racemic tocopherol being particularly preferred for TPGS.

In another embodiment, the moiety L¹-Y¹ has a structure according to the following formula:

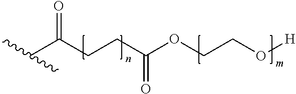

wherein n is selected from 1 to 20, m is selected from 1 to 5000. In another embodiment, n is 4. In another embodiment, m is a selected from 1 to 2,500.

Methods of making the above solubilizing agents are known in the art as disclosed in U.S. Pat. Nos. 6,045,826, 6,191,172, 6,632,443 and WO 96/17626, all herein incorporated by reference in their entirety. Similarly, TPGS may be prepared accordingly, or by using succinic anhydride as the linker in place of the diacid chloride as precursor to the four-carbon linker.

In another embodiment, the formulations of the present application include from about 10% to about 50% by weight of a solubilizing agent, such as TPGS or TPGS-1000. The formulations include from about 15% to about 40% (w/w) solubilizing agent, from about 20% to about 40% (w/w), and from about 20 to about 35% (w/w). In another embodiment, the present application includes from about 0.01% (w/w) to about 5% (w/w), from about 0.01% (w/w) to about 0.1% (w/w), from about 0.01% (w/w) to about 1% (w/w), from about 0.1% (w/w) to about 1% (w/w), from about 0.1% (w/w) to about 0.75% (w/w), 1% (w/w) to about 3% (w/w), and from about 0.05% (w/w) to about 0.25% (w/w) of a solubilizing agent.

The soft gel capsules of the present application (based on a soft gel capsule weight of from about 900 mg to about 1200 mg) include a solubilizing agent from about 1% to about 30% by weight. In one embodiment, the soft gel capsule includes from about 1%, 3%, or 5% to about 30% (w/w), from about 8% to about 20% of a solubilizing agent, such as Solutol HS 15, Cremophor EL, TPGS or TPGS-1000.

### Water-Soluble Reducing Agent or Lipophilic Reducing Agent:

In another embodiment, the water-soluble reducing agent is vitamin C, a water-soluble vitamin C derivative (e.g., a salt), or a combination thereof. In one embodiment, the compositions of the present application are selected from ascorbic acid (vitamin C), a vitamin C derivatives, salts thereof and combinations thereof. In one embodiment, the vitamin C salt, or salt of a vitamin C derivative is an edible (e.g., pharmaceutically acceptable) salt, such as a calcium, sodium, magnesium, potassium and zinc salt. Mixed salts of vitamin C or a vitamin C derivative are also within the scope of the present application. The compositions may include one or more vitamin C derivative. The vitamin C derivative can be any analog of vitamin C. Exemplary vitamin C derivative include those in which at least one of the hydroxyl groups of the ascorbic acid molecule (e.g., 2-OH, 3-OH, 5-OH, 6-OH) is derivatized with a modifying group (see e.g., U.S. Pat. No. 5,078,989 to Ando et al.). Alternatively one or more of the hydroxyl group can be substituted with another moiety. In another embodiment, the compositions may include vitamin C as well as at least one vitamin C derivative.

Exemplary vitamin C derivatives according to this embodiment include esters, such as 6-O-octanoyl-ascorbic acid, 6-O-dodecanoyl-ascorbic acid, 6-O-tetradecanoyl-ascorbic acid, 6-O-octadecanoyl-ascorbic acid, 6-O-dodecanedioyl-ascorbic acid, 6-O-docosanedioyl-ascorbic acid, 6-O-thapsoyl-ascorbic acid, 6-O-suberoyl-ascorbic acid, 6-O-adipoyl-ascorbic acid. Other examples include those esters, in which the lipophilic part of the molecule represents a mono- or polyunsaturated fatty acid. In one embodiment, the unsaturated fatty acids is an essential fatty acids associated with a health benefit (e.g., human health), such as an omega-3 (alpha-linolenic acid), omega-6 or omega-9 fatty acid. Other examples include esters of vitamin C including an amino acid residue. In another embodiment, the compositions of the present application include 2-O-alkyl or 3-O-alkyl derivatives of vitamin C. 3-O-alkyl-ascorbic acids have been reported by Nihro et al., Chem. Pharm. Bull. 1991, 39: 1731-1735, the disclosure of which is incorporated herein by reference. In yet another embodiment, the vitamin C derivative is a glucoside of ascorbic acid, such as ascorbic acid 1-glucoside, ascorbic acid 2-glucoside, ascorbic acid 3-glucoside, ascorbic acid 5-glucoside, and ascorbic acid 6-glucoside. Examples include 2-O-(alpha-D-glucopyranosyl)-ascorbic acid (see e.g., U.S. Pat. No. 5,137,723) and 2-O-(beta-D-glucopyranosyl)-ascorbic acid (see e.g., U.S. Patent Application No. 2005/0113312). Also within the scope of the present application are difunctionalized derivatives of vitamin C, such as e.g., 6-O-acyl-2-O-(alpha-D-glucopyranosyl) ascorbic acids (see e.g., Yamamoto et al., J. Med. Chem. 2002, 45(2): 462-468. The above references are incorporated herein by reference. In a further embodiment, the vitamin C derivative is a phosphate of ascorbic acid. In another embodiment the ascorbyl phosphate is a salt of an alkali metal, an alkaline earth metal, or a transition metal. Preferred examples include magnesium ascorbyl phosphate, sodium ascorbyl phosphate (e.g., sodium salt of ascorbyl-2-monophosphate), calcium ascorbyl phosphate, potassium ascorbyl phosphate and mixed salts, such as e.g., sodium magnesium ascorbyl phosphate or sodium calcium ascorbyl phosphate, aminopropyl ascorbyl phosphate. The ascorbyl phosphate can exist as a hydrate, wherein dihydrates are common. An exemplary dihydrate is available for example from DSM under the product name STAY-C 50.

In another embodiment of the formulation, the stabilizer is in excess in relation to the POFA, or the POFA is in excess of the stabilizer. In another exemplary embodiment, the ratio of the POFA to the stabilizer is from about 1:1 (w/w) to about 1:6 (w/w), from about 1:1 (w/w) to about 1:5 (w/w), from about 1:1.3 (w/w) to about 1:3 (w/w), from about 1:2 (w/w) to about 1:4 (w/w), or about 1:3 (w/w). In another embodiment, the ratio of the stabilizer to the POFA is from about 1:1 (w/w) to about 1:6 (w/w), from about 1:1 (w/w) to about 1:5 (w/w), from about 1:1.3 (w/w) to about 1:3 (w/w), from about 1:2 (w/w) to about 1:4 (w/w), or about 1:3 (w/w).

In another embodiment, the stabilizer is vitamin C or a vitamin C derivative. In one example, the vitamin C or the vitamin C derivative is used in a molar excess in relation to the POFA. In another exemplary embodiment, the ratio of the POFA to vitamin C or vitamin C derivative is from about 1:1 (w/w) to about 1:6 (w/w), from about 1:1 (w/w) to about 1:10 (w/w), from about 1:1.3 (w/w) to about 1:5 (w/w), from about 1:2 (w/w) to about 1:4 (w/w), or about 1:3 (w/w).

### The Metal Chelating Agent:

In another embodiment, the metal chelator, chelating agent or metal chelating moiety is a chelator that has demonstrated affinity metal ions. Such metal ions include certain metal ions such as iron, but may also include lead, mercury and nickel. In one aspect, the chelator is EDTA or ethylenediaminetetraacetic acid disodium salt dihydrate and the metal ion is iron (II) or iron (III). In one aspect, the metal ion is iron (III). In one embodiment, the formulations of the present application include from about 0.001% to about 0.01% by weight of the chelator relative to the POFA (w/w), (i.e. weight of chelator/weight of POFA), from about 0.01% to about 0.1%, from about 0.1% to about 0.5%, from about 0.5% to about 1.0%, from about 1.0% to about 2.0%, from about 2.0% to about 4.0%, from about 4.0% to about 6.0%, or about 4% of the chelator relative to the POFA. In another embodiment, the formulations of the present application include from about 6.0% to about 10.0% by weight of the chelator relative to the POFA (w/w), from 10.0% to about 15%, or from about 15% to about 20% by weight of the chelator relative to the POFA.

### The Bisulfite Agent:

In one embodiment, the bisulfite agent of the present formulation is a metal bisulfite. In one aspect, the bisulfite agent is sodium bisulfite. The sodium bisulfite will react with any aldehyde present in the formulation to form a bisulfite addition compound and eliminates any undesired aldehyde odors. In one embodiment, the formulations of the present application include from about 0.0001% to about 0.001% by weight of sodium bisulfite relative to the POFA (w/w), (i.e. weight of sodium bisulfite/weight of POFA), from about 0.001% to about 0.01%, from about 0.01% to about 0.05%, from about 0.05% to about 0.10%, from about 0.10% to about 0.2%, from about 0.2% to about 0.4%, from about 0.4% to about 0.6%, or about 0.5% of sodium bisulfite relative to the POFA. In another embodiment, the formulations of the present application include from about 0.6% to about 1.0% by weight of the chelator relative to the POFA (w/w), from 1.0% to about 1.5%, or from about 1.5% to about 2.0% by weight of sodium bisulfite relative to the POFA. As one skilled in the art would appreciate, compositions comprising the formulation that is known or that is determined to contain larger concentrations of metals, such as iron, will require the use of higher concentrations of the metal bisulfite, and the concentration of the metal bisulfite may be adjusted accordingly.

### Other Components:

The formulations described herein (either aqueous or non-aqueous) can further include various ingredients useful to stabilize the composition, promote the bioavailability of the lipophilic bioactive molecule, such as the POFA, or provide nutritional value. Exemplary additives of the present formulations include, without limitation, one or more alternative solubilizing agents, pharmaceutical drug molecules, antibiotics, sterols, vitamins, provitamins, carotenoids (e.g., alpha and beta-carotenes, cryptoxanthin, lutein and zeaxanthin), phospholipids, L-carnitine, starches, sugars, fats, stabilizers, reducing agents, free radical scavengers, amino acids, amino acid analogs, proteins, solvents, emulsifiers, adjuvants, sweeteners, fillers, flavoring agents, coloring agents, lubricants, binders, moisturizing agents, preservatives, suspending agents, starch, hydrolyzed starch(es), derivatives thereof and combinations thereof.

In one embodiment, the formulation further comprises gelatin. In another embodiment, the formulation further comprises sorbitol, glycerin, or any ester derivatives therefrom. In another embodiment, the formulation further comprises polysorbate 80, hydroxylated lecithin, medium chain triglycerides, annato seed extract or soybean oil and mixtures thereof. In another embodiment, the formulation further comprises omega-3 enriched hill oil. In yet another embodiment, the formulation further comprises rice bran oil, carrotenoids, titanium dioxide, suspending agents such as silica (silicon dioxide) or riboflavin and mixtures thereof. Various other additives can be incorporated into the present formulations including, without limitation, phospholipids, L-carnitine, anti-inflammatory agents, anti-aging agents, starches, sugars, fats, stabilizers, amino acids, proteins, flavorings, coloring agents, hydrolyzed starch(es) and derivatives thereof (such as time release esters (Ester-C, Ester-E)) or combinations thereof. Anti-inflammatory agents of use in the present application include, but are not limited to, bisabolol, mentholatum, dapsone, aloe, hydrocortisone, and the like. Anti-aging agents of use in the present application include, but are not limited to, niacinamide, retinol and retinoid derivatives, AHA, lipoic acid, beta hydroxy acids, salicylic acid, copper binding peptides and the like.

Vitamin(s) in a unit dosage form of the present application are present in amount ranging from about 5 mg to about 500 mg. More particularly, the vitamin(s) is present in an amount ranging from about 10 mg to about 400 mg. Even more specifically, the vitamin(s) is present from about 250 mg to about 400 mg. Most specifically, the vitamin(s) is present in an amount ranging from about 10 mg to about 50 mg. For example, B vitamins are in usually incorporated in the range of about 1 milligram to about 10 milligrams, i.e., from about 3 micrograms to about 50 micrograms of B12. Folic acid, for example, is generally incorporated in a range of about 50 to about 400 micrograms, biotin is generally incorporated in a range of about 25 to about 700 micrograms and cyanocobalamin is incorporated in a range of about 3 micrograms to about 50 micrograms.

Mineral(s) in a unit dosage form of the present application are present in an amount ranging from about 25 mg to about 1000 mg. More particularly, the mineral(s) are present in the composition ranging from about 25 mg to about 500 mg. Even more particularly, the mineral(s) are present in the composition in an amount ranging from about 100 mg to about 600 mg. In the formulations of the present application the additional components are usually a minor component (from about 0.001% to about 20% by weight or preferably from about 0.01% to about 10% by weight) with the remainder being various vehicles or carriers and processing aids helpful for forming the desired dosing form.

### Pharmaceutical Formulations:

According to another aspect, the present application provides pharmaceutical formulations comprising a formulation of the present application and a pharmaceutically acceptable carrier. Pharmaceutical formulations include nutraceutical formulations. An exemplary unit dosage form (e.g., contained in a soft gel capsule) of the present application includes a pharmaceutical grade lipophilic bioactive molecule (e.g., POFA comprising an omega-3-fatty acid, DHA) in an amount of about 1% to about 30% by weight. In one embodiment, the unit dosage form (e.g., soft gel capsule) includes from about 3% to about 20% (w/w), or from about 5% to about 20% of a lipohilic bioactive molecule. Typically, soft-gel formulations include from about 5% to about 30% (w/w) of lipophilic bioactive molecule, from about 15% to about 40% (w/w) solubilizing agent (e.g., TPGS or TPGS-1000), from about 30% to about 60% (w/w) lipophilic carrier (e.g., krill oil or POFA) and from about 1% to about 10% (w/w) viscosity enhancer (e.g., beeswax). In another embodiment, the soft gel capsule of the present application includes phospholipids comprising omega-3-fatty acids (POFA), vitamin C, solubilizing agent (e.g., Solutol HS 15, Cremophor EL, TPGS or TPGS-1000 or mixtures thereof), beeswax and a lipophilic carrier (e.g., hill oil) enriched with omega fatty acids. In another embodiment, the omega fatty acids are combined with a solubilizing agent useful to improve the bioavailability of the omega fatty acids. Such formulations may further contain additional active ingredients and/or pharmaceutically or cosmetically acceptable additives or vehicles, including solvents, adjuvants, excipients, sweeteners, fillers, colorants, flavoring agents, lubricants, binders, moisturizing agents, preservatives and mixtures thereof. The formulations may be suitable for topical (e.g., a cream, lotion, gel, ointment, dermal adhesive patch), oral (e.g., a soft gel, capsule, tablet, caplet, granulate), or parenteral (e.g., suppository, sterile solution) administration. Among the acceptable vehicles and solvents that may be employed for administration by injection are water, mildly acidified water (e.g. acidified carbonated water), Ringer's solution and isotonic sodium chloride solution. In some embodiments, the formulation is in the form of a drinkable liquid or syrup and can be formulated in a mildly acidified water (e.g. acidified carbonated water) as the carrier. The POFA, when combined with a solubilizing agent of the present application, can be administered to a warm-blooded animal, particularly a human, in need of the prophylaxis or therapy. The method comprises administering to such human or warm-blooded animal, an effective amount of a water-soluble formulation of the present application. When the hydrophobic moiety of the solubilizing agent is linked to the hydrophilic moiety through a linker, which is cleavable in vivo, the formulation can provide an additional benefit for the patient. In vivo, the solubilizing agent is hydrolyzed by enzymes and is systemically converted back to the respective tocopherol with concomitant release of the omega-3-fatty acids.

The pharmaceutical composition can be prepared according to known methods. Formulations are described in detail in a number of sources, which are well known and readily available to those skilled in the art. For example, Remington's Pharmaceutical Science by E. W. Martin describes formulation, which can be used in connection with the subject present application. In accordance with the present application, pharmaceutical compositions are provided which comprise, an active ingredient as described, supra, and an effective amount of one or more pharmaceutically acceptable excipients, vehicles, carriers or diluents. Further, acceptable carriers can be either solid or liquid. Solid form preparations include powders, tablets, pills, capsules, cachets, suppositories and dispersible granules. A solid carrier can be one or more substances, which may act as diluents, flavoring agents, solubilizing agents, lubricants, suspending agents, binders, preservatives, tablet disintegrating agents or encapsulating materials.

For oral administration, the pharmaceutical compositions can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets can be coated by methods well known in the art. Liquid preparations for oral administration can take the form of, for example, solutions, syrups or suspensions, or they can be presented as a dry product for constitution with water or other suitable vehicle before use. The preparations can also contain buffer salts, flavoring, coloring and sweetening agents as appropriate. For buccal administration, the compositions can take the form of tablets or lozenges formulated in conventional manner.

The disclosed pharmaceutical compositions can be subdivided into unit doses containing appropriate quantities of the active component. The unit dosage form can be a packaged preparation, such as packeted tablets, capsules, and powders in paper or plastic containers or in vials or ampoules. Also, the unit dosage can be a liquid based preparation or formulated to be incorporated into solid food products, chewing gum, or lozenges. Pharmaceutically acceptable salts (counter ions) can be conveniently prepared by ion-exchange chromatography or other methods as are well known in the art. The formulations of the present application can take a variety of forms adapted to the chosen route of administration. Those skilled in the art will recognize a wide variety of non-toxic pharmaceutically acceptable solvents that may be used to prepare solvates of the compounds of the present application, such as water, ethanol, propylene glycol, mineral oil, vegetable oil and dimethylsulfoxide (DMSO).

The compositions of the present application may be administered orally, topically, parenterally or rectally in dosage unit formulations containing conventional non-toxic pharmaceutically acceptable carriers, adjuvants and vehicles. It is further understood that the best method of administration may be a combination of methods. The term parenteral as used herein includes subcutaneous injections, intradermal, intravascular (e.g., intravenous), intramuscular, spinal, intrathecal injection or like injection or infusion techniques. The formulations are in a form suitable for oral use, for example, as tablets, troches, lozenges, aqueous or oily suspensions, dispersible powders or granules, emulsion, hard or soft capsules, soft gel capsules, or syrups or elixirs. The formulations described herein may be prepared according to any method known in the art for the manufacture of pharmaceutical formulations and nutraceuticals, and such compositions may contain one or more agents selected from the group consisting of sweetening agents, flavoring agents, coloring agents and preserving agents in order to provide pharmaceutically elegant and palatable preparations. Tablets may contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil. Aqueous suspensions contain the active materials in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; and dispersing or wetting agents, which may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide palatable oral preparations. These compositions may be preserved by the addition of an anti-oxidant such as ascorbic acid. Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

In one embodiment, the formulations of the present application may also be in the form of oil-in-water emulsions and water-in-oil emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth; naturally-occurring phosphatides, for example soy bean, lecithin, and esters or partial esters derived from fatty acids and hexitol; anhydrides, for example sorbitan monooleate; and condensation products of the said partial esters with ethylene oxide, for example polyoxyethylene sorbitan monooleate. The emulsions may also contain sweetening and flavoring agents. Syrups and elixirs may be formulated with sweetening agents, for example glycerol, propylene glycol, sorbitol or sucrose. Such formulations may also contain a demulcent, a preservative, and flavoring and coloring agents. The formulations may be in the form of a sterile injectable aqueous or oleaginous suspension. This suspension may be formulated according to the known art using those suitable dispersing or wetting agents and suspending agents, which have been mentioned above. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

For administration to non-human animals, the formulations of the present application may be added to the animal's feed or drinking water. Also, it will be convenient to formulate animal feed and drinking water products so that the animal takes in an appropriate quantity of the compound in its diet. It will further be convenient to present the compound in a composition as a premix for addition to the feed or drinking water. The composition can also be added as a food or drink supplement for humans. Dosage levels (with respect to lipophilic bioactive molecule) of the order of from about 1 mg to about 250 mg per kilogram of body weight per day are useful. For example, a dosage level from about 25 mg to about 150 mg per kilogram of body weight per day, are useful. Dosage unit forms will generally contain between from about 1 mg to about 500 mg of the POFA (e.g., comprising omega fatty acids, omega-3-fatty acids (e.g., ALA, DHA)) and carotenoids (e.g., astaxanthin, fucoxanthin, cantaxanthin and the like). For example, dosage unit forms of about 1 mg to about 250 mg, about 1 mg to about 100 mg or 1 mg to about 80, 60, 40, 20 or 10 mg are useful. Frequency of dosage may also vary depending on the compound used and the particular disease treated. It will be understood, however, that the specific dose level for any particular patient will depend upon a variety of factors including the activity of the specific compound employed, the age, body weight, general health, sex, diet, time of administration, route of administration and rate of excretion, drug combination and the severity of the particular disease undergoing therapy. The present application also provides packaged formulations and instructions for use of the tablet, capsule, soft gel capsule, elixir, etc. Typically, the dosage requirement is between about 1 to about 4 dosages a day.

### Exemplary Formulations Including Stabilizers:

In another embodiment, the present application provides a formulation which comprises: (a) a POFA; (b) a solubilizing agent (e.g., Solutol HS 15, Cremophor EL, TPGS etc. . . . ); (c) a water-soluble reducing agent (stabilizer) (e.g., vitamin C, a vitamin C derivative or mixtures thereof); (d) EDTA; and (e) sodium bisulfite. In another embodiment, the ratio of the POFA to the solubilizing agent is from about 1:0.3 (w/w) to about 1:20 (w/w), from about 1:1 (w/w) to about 1:20 (w/w), from about 1:1 (w/w) to about 1:10 (w/w), from about 1:1.3 (w/w) to about 1:5 (w/w), from about 1:2 (w/w) to about 1:4 (w/w), about 1:3 (w/w); from about 1:0.3 (w/w) to about 1:1 (w/w), or from about 1:0.5 (w/w) to about 1:2 (w/w). In another embodiment, the ratio of the POFA to the TPGS is from about 1:2 to about 1:4, or about 1:3. In another embodiment, the ratio of the POFA to the TPGS is from about 1:2 to about 1:4, or about 1:3.

In another embodiment, the present application provides a formulation which comprises: (a) a POFA; (b) a solubilizing agent (e.g., Solutol HS 15, Cremophor EL, TPGS or PTGS-1000); (c) vitamin C, a vitamin C derivative, or combinations thereof; (d) EDTA, and (e) sodium bisulfite. In one embodiment, the POFA is present in the formulation in an amount of at least about 0.5% by weight, at least about 1% by weight, at least about 1.5% by weight, at least about 2% by weight, at least about 2.5% by weight, at least about 3% by weight, at least about 3.5% by weight, at least about 4% by weight, at least about 4.5% by weight or at least about 5% by weight. In another embodiment, the POFA is present in the formulation in an amount of at least about 95% by weight, at least about 96% by weight or at least about 97% by weight.

In another embodiment, the present application provides a formulation which comprises: (a) a POFA; (b) a solubilizing agent (e.g., Solutol HS 15, Cremophor EL, TPGS or TPGS-1000); (c) a stabilizer; (d) EDTA, and (e) sodium bisulfite. In another embodiment, the present application provides a formulation which comprises: (a) a POFA; (b) a solubilizing agent; (c) Vitamin C, a Vitamin C derivative, or combinations thereof; (d) EDTA, and (e) sodium bisulfite. In another embodiment, the present application provides a formulation which comprises: (a) a POFA; (b) Solutol HS 15, Cremophor EL, TPGS or TPGS-1000; (c) Vitamin C, a Vitamin C derivative, or combinations thereof; (d) EDTA, and (e) sodium bisulfite. In another embodiment, the present application provides a formulation which comprises: (a) a POFA; (b) a solubilizing agent (e.g., Solutol HS 15, Cremophor EL, TPGS or TPGS-1000); (c) a stabilizer; (d) EDTA, and (e) sodium bisulfite. In another embodiment, the present application provides a formulation which comprises: (a) a POFA; (b) a solubilizing agent; (c) Vitamin C, a Vitamin C derivative, or combinations thereof. As provided throughout the present application, unless specified otherwise, the use of the solubilizing agent, even when exemplified by the phrase “e.g., TWEEN-85, TPGS or TPGS-1000” for example, may include each of the disclosed solubilizing agents individually, and their mixtures thereof.

In one embodiment, the present application provides a formulation which comprises: (a) a POFA comprising oleic acid; (b) a solubilizing agent; (c) a stabilizer; (d) EDTA, and (e) sodium bisulfite. In another embodiment, the present application provides a formulation which comprises: (a) a POFA comprising oleic acid; (b) a solubilizing agent; and (c) Vitamin C, a Vitamin C derivative, or combinations thereof; (d) EDTA, and (e) sodium bisulfite. In another embodiment, the present application provides a formulation which comprises: (a) a POFA comprising oleic acid; (b) Solutol HS 15, Cremophor EL, TPGS or TPGS-1000; and (c) Vitamin C, a Vitamin C derivative, or combinations thereof; (d) EDTA, and (e) sodium bisulfite. In another embodiment, the formulation comprises: (a) a POFA comprising gamma linolenic acid; (b) a solubilizing agent; (c) a stabilizer; (d) EDTA, and (e) sodium bisulfite. In another embodiment, the formulation comprises: (a) a POFA comprising gamma linolenic acid; (b) a solubilizing agent; (c) Vitamin C, a Vitamin C derivative, or combinations thereof; (d) EDTA, and (e) sodium bisulfite. In another embodiment, the formulation comprises: (a) a POFA comprising gamma linolenic acid; (b) TPGS-1000; (c) Vitamin C, a Vitamin C derivative, or combinations thereof; (d) EDTA, and (e) sodium bisulfite. In another embodiment, the formulation comprises: (a) a POFA comprising docosahexaenoic acid; (b) a solubilizing agent (e.g., Solutol HS 15, Cremophor EL, TPGS or TPGS-1000); (c) a stabilizer; (d) EDTA, and (e) sodium bisulfite. In another embodiment, the formulation comprises: (a) a POFA comprising docosahexaenoic acid; (b) a solubilizing agent; (c) Vitamin C, a Vitamin C derivative, or combinations thereof; (d) EDTA, and (e) sodium bisulfite. In another embodiment, the formulation comprises: (a) a POFA comprising docosahexaenoic acid; (b) Solutol HS 15, Cremophor EL, TPGS or TPGS-1000; (c) Vitamin C, a Vitamin C derivative, or combinations thereof; (d) EDTA, and (e) sodium bisulfite. In one embodiment, the formulation includes from about 0.01% (w/w) to about 5% (w/w) of docosahexaenoic acid. In another embodiment, the formulation includes from about 0.01% (w/w) to about 0.1% (w/w) of docosahexaenoic acid. In another embodiment, the formulation includes from about 0.01% (w/w) to about 1% (w/w) of docosahexaenoic acid. In another embodiment, the formulation includes from about 0.1% (w/w) to about 1% (w/w) of docosahexaenoic acid. In another embodiment, the formulation includes from about 0.1% (w/w) to about 0.75% (w/w) of docosahexaenoic acid. In another embodiment, the formulation includes from about 1% (w/w) to about 3% (w/w) of docosahexaenoic acid. In another embodiment, the formulation includes from about 0.05% (w/w) to about 0.25% (w/w) of docosahexaenoic acid. In another embodiment, the formulation comprises: (a) a POFA comprising eicosapentaenoic acid; (b) a solubilizing agent (e.g., TWEEN-85, TPGS or TPGS-1000); (c) a stabilizer; (d) EDTA, and (e) sodium bisulfite. In another embodiment, the formulation comprises: (a) a POFA comprising eicosapentaenoic acid; (b) a solubilizing agent; (c) Vitamin C, a Vitamin C derivative, or combinations thereof; (d) EDTA, and (e) sodium bisulfite. In another embodiment, the formulation comprises: (a) a POFA comprising eicosapentaenoic acid; (b) TWEEN-85, Solutol HS 15, Cremophor EL, TPGS or TPGS-1000; (c) Vitamin C, a Vitamin C derivative, or combinations thereof; (d) EDTA, and (e) sodium bisulfite. In another embodiment, the formulation comprises from about 0.01% (w/w) to about 5% (w/w) of eicosapentaenoic acid; about 0.01% (w/w) to about 0.1% (w/w); about 0.01% (w/w) to about 1% (w/w); about 0.1% (w/w) to about 1% (w/w); about 0.1% (w/w) to about 0.75% (w/w); 1% (w/w) to about 3% (w/w); and about 0.05% (w/w) to about 0.25% (w/w) of eicosapentaenoic acid.

### Methods of Making the Formulations:

The present application also provides methods (e.g., processes) of making the formulations and compositions of the present application. In one embodiment, the POFA, solubilizing agent and reducing agent (e.g., vitamin C or a water-soluble vitamin C derivative), EDTA, and sodium bisulfite, and optionally other components of the formulation are placed in a container. A solvent is then added and the mixture is optionally heated, thereby dissolving the components and forming the formulation. In another exemplary embodiment, the POFA is dissolved in a solvent optionally using heat. The solubilizing agent, the reducing agent (e.g., vitamin C or a water-soluble vitamin C derivative), EDTA, and sodium bisulfite and optionally other components are added to the above solution creating a mixture, which is stirred and optionally heated to dissolve all components in the mixture, thus creating the formulation. In another embodiment, a solubilizing agent is dissolved in a solvent (e.g., water). The POFA, the reducing agent (e.g., vitamin C or a water-soluble vitamin C derivative), EDTA, and sodium bisulfite, together with any optional components are added and dissolved in the above solution (optionally using heat), thus creating the formulation. In another exemplary embodiment, the reducing agent (e.g., vitamin C or a water-soluble vitamin C derivative) is dissolved in a solvent of choice. The POFA and the solubilizing agent, EDTA, and sodium bisulfite together with any optional components are added and are dissolved in the solution (optionally using heat), thus creating the formulation.

### Exemplary Processes:

In a particular example, the solubilizing agent is as disclosed herein. In one embodiment, the solubilizing agent used in the methods of the present application is TWEEN-85, Solutol HS 15, Cremophor EL, TPGS or TPGS-1000 or mixtures thereof. In one example, the POFA is solubilized in the above emulsion in the form of micelles that are formed between the POFA and the solubilizing agent. In one example, the micelles have a median particle size of less than about 60 nm (e.g., between about 10 and about 30 nm). In one example, the present application provides a POFA stock solution, which is prepared by a method according to any of the above embodiments. In one example, the above water-soluble POFA stock solution can be used to prepare a beverage of the present application. In one embodiment, the above method further includes contacting the water-soluble POFA stock solution with an original beverage to form a POFA beverage of the present application. Exemplary original beverages useful in the methods of the present application are disclosed herein. Exemplary lipophilic bioactive molecules, which can be stabilized using any of the above methods include POFA, omega-3-fatty acids (e.g., docosahexaenoic acid (DHA), eicosapentaenoic acid (EPA) and alpha-linolenic acid (ALA)), omega-6-fatty acid, omega-9-fatty acid, essential oils, flavor oils and lipophilic vitamins; and mixtures thereof.

In one example, the amount of water-soluble reducing agent that is contacted with the above emulsion is equivalent to an over-stoichiometric mole ratio with respect to the POFA. In another example, the amount is equivalent to a ratio of POFA to water-soluble reducing agent of about 1:1 to about 1:10 (w/w); about 1:1 to about 1:8 (w/w), about 1:1 to about 1:6 (w/w) or about 1:1 to about 1:4 (w/w), or about 1:1 to about 1:3 (w/w).

### Additives or Carriers for Stabilized Surfactants and POFA:

The pre-drying emulsion (or emulsion) of the present application may include about 0.1% by weight to about 99% by weight additive or carrier, wherein the additive or carrier may also include a sweetener, a flavoring agent, a coloring agent, an anti-foaming agent, a nutrient, calcium or a calcium derivative, an energy-generating additive, an herbal supplement, a concentrated plant extract, a preservative, and/or combinations thereof.

In one aspect, the additive or carrier may include a gum and maltodextrin. In another aspect, the additive may be selected from the group consisting of crystalline cellulose, α-cellulose cross-linked carboxymethyl cellulose sodium, cross-linked starch, gelatin, casein, gum tragacanth, polyvinylpyrrolidone, chitin, chitosan, dextrin, kaolin, silicon dioxide hydrate, colloidal silicon dioxide, light silica, synthetic aluminum silicate, synthetic hydrotalcite, titanium oxide, dry aluminum hydroxy gel, magnesium carbonate, calcium carbonate, precipitated calcium carbonate, bentonite, aluminum magnesium metasilicate, calcium lactate, calcium stearate, calcium hydrogen phosphate, phosphoric acid anhydride, calcium hydrogen and talc. In one aspect, the additive comprises flowing agents selected from silicon dioxide and titanium oxide that promotes flowability or powdery characteristics of the dry powder. In one aspect, the emulsion comprises one or more additives selected from the group consisting of crystalline cellulose, α-cellulose, cross-linked carboxymethyl cellulose sodium, cross-linked starch, gelatin, casein, gum tragacanth, chitin, chitosan, calcium hydrogen phosphate, calcium hydrogen and precipitated calcium carbonate, and combinations thereof. In another aspect, the additive is comprised of wetting agents to assist in the dissolution of the dry powder, when the dry powder is dissolved in water. Such agents may include lecithin and the like.

In another aspect, the additives may include polymers that are added in an amount such that, where desired, the solution resulting from the re-dissolved powder of the present application remains stable over a period of at least 6 months or 12 months. The additive may include cellulosic polymers. Exemplary cellulosic polymers that may be used include hydroxypropyl methyl cellulose acetate, hydroxypropyl methyl cellulose, hydroxypropyl cellulose, methyl cellulose, hydroxyethyl methyl cellulose, hydroxyethyl cellulose acetate and hydroxyethyl ethyl cellulose. In another aspect, the polymers may include hydroxypropyl methyl cellulose and hydroxypropyl cellulose acetate. In another aspect, the polymers contain at least one ionizable substituent, which may be either ether-linked or ester-linked. Exemplary ether-linked ionizable substituents include: carboxylic acids, such as acetic acid, propionic acid, benzoic acid, salicylic acid, alkoxybenzoic acids such as ethoxybenzoic acid or propoxybenzoic acid, the various isomers of alkoxyphthalic acid such as ethoxyphthalic acid and ethoxyisophthalic acid, the various isomers of alkoxynicotinic acid such as ethoxynicotinic acid, etc.

In another aspect, exemplary cellulosic polymers may include hydroxypropyl methyl cellulose acetate succinate, hydroxypropyl methyl cellulose succinate, hydroxypropyl cellulose acetate succinate, hydroxyethyl methyl cellulose succinate, hydroxyethyl cellulose acetate succinate, hydroxypropyl methyl cellulose phthalate, hydroxyethyl methyl cellulose acetate succinate, hydroxyethyl methyl cellulose acetate phthalate, carboxyethyl cellulose, carboxymethyl cellulose, carboxymethyl ethyl cellulose, ethyl carboxymethyl cellulose, cellulose acetate phthalate, methyl cellulose acetate phthalate, ethyl cellulose acetate phthalate, hydroxypropyl cellulose acetate phthalate, hydroxypropyl methyl cellulose acetate phthalate, hydroxypropyl cellulose acetate phthalate succinate, hydroxypropyl methyl cellulose acetate succinate phthalate, hydroxypropyl methyl cellulose succinate phthalate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetate isophthalate, cellulose acetate pyridinedicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid cellulose acetate, and ethyl picolinic acid cellulose acetate. In another aspect, the cellulosic polymers may contain a non-aromatic carboxylate group, such as hydroxypropyl methyl cellulose acetate succinate, hydroxypropyl methyl cellulose succinate, hydroxypropyl cellulose acetate succinate, hydroxyethyl methyl cellulose acetate succinate, hydroxyethyl methyl cellulose succinate, hydroxyethyl cellulose acetate succinate and carboxymethyl ethyl cellulose.

Where it is desired to provide coloring pigments to the formulation (emulsions, powders and solutions), various pigments may be added to the formulation, as are known in the art.

In one embodiment, flavor and/or fragrance ingredients or additives may be added to the formulation. As used herein, the terms “flavor” and/or “fragrance ingredient or additives” refer to a variety of flavor and fragrance materials of both natural and synthetic origin. Such materials may include single compounds and mixtures of compounds. Specific examples of such additives may be found in, e.g., in Fenaroli's Handbook of Flavor Ingredients, 1975, CRC Press; Synthetic Food Adjuncts, 1947 by M. B. Jacobs, edited by Van Nostrand; or Perfume and Flavor Chemicals by S. Arctander 1969, Montclair, N.J. (USA). These materials and substances are well known to one of skill in the art of perfuming, flavoring, and/or aromatizing consumer products to imparting an odor and/or a flavor or taste to a product, or to modify the odor and/or taste of the product.

Examples of the perfumes mentioned above include peppermint oil, beefsteak plant oil, spearmint oil, lavender oil, rosemary oil, cumin oil, clove oil, eucalyptus oil, lemon oil, orange oil, lime oil, rose oil, cinnamon oil, pepper oil, vanilla, ginger oil, and the like. Examples of the spices mentioned above include spices extracted from capsicum, cardamon, mints, peppers, turmeric, cumin, sage, parsley, oregano, saffron, rosemary, thyme, and the like.

In one embodiment, the composition further comprises an additive such as a sugar or sugar derivative, such as sucrose, glucose, lactose, levulose, fructose, maltose, ribose, dextrose, isomalt, sorbitol, mannitol, xylitol, lactitol, maltitol, pentatol, arabinose, pentose, xylose and galactose, and combinations thereof. Typically, the compositions of the present application may comprise from 0.01 to 10% by weight, about 10% to 25% by weight, or about 25% to 50% by weight of the above additive, relative to the weight of the dried powder formulation.

In one embodiment, the additives including coloring pigments, perfumes, flavoring and spices may be used in the appropriate concentration to obtain the desired color, flavors, aroma, taste and ultimate clarity of solution.

### Drying of Stabilized Surfactants and POFA (or Krill Oil) Emulsions:

One aspect of the drying method for the stabilized emulsion includes a spray drying method. The spray-drying method may include, for example, a method for spraying from a high-pressure nozzle. In another aspect, the method for spray-drying uses a centrifugal force, such as an atomizer. The gas or air that may be used for the spray drying includes heated air or hot air at a temperature sufficient to dry the powder having the desired moisture content. In one aspect, the gas is an inert gas such as nitrogen or nitrogen-enriched air. In one aspect, the hot gas temperature may be at about 50° C. to 300° C., from about 60° C. to 100° C., from about 60° C. to 250° C., from about 75° C. to about 185° C., from about 100° C. to about 180° C., about 180° C. to about 190° C., or about 180° C. The high pressure that may be used for the spray during process used in a high pressure nozzle may include about 10 to 1,000 psi, about 100 to about 800 psi, about 200 to about 500 psi. The spray drying may be carried out under conditions such that the residual water or residual moisture content of the dry powder may be controlled to about 1% to about 6%, about 1% to about 5%, about 2% to about 6%, about 3% to about 6%, about 3% to about 5%. According to the present method, without being bound by any particular theory presented herein, it is determined that lower moisture content or higher moisture content than the desired ranges using the present methods as described herein, results in a powder composition that may lose its ability to re-dissolve in water, resulting in solutions that are cloudy and not clear. On the other hand, it was determined that higher residual moisture of the dry powder than the above ranges obtained by the present methods provides powder formulations that may coagulate.

In one aspect, the emulsions may then be sprayed dried in conventional spray drying equipment from commercial suppliers, such as Buchi, Niro, Yamato Chemical Co., Okawara Kakoki Co., and similar commercially available spray drier. Spray drying processes, such as rotary atomization, pressure atomization and two-fluid atomization may also be used. Examples of the devices used in these processes include Parubisu Mini-Spray GA-32 and Parubisu Spray Drier DL-41 (Yamato Chemical Co.) or Spray Drier CL-8, Spray Drier L-8, Spray Drier FL-12, Spray Drier FL-16 or Spray Drier FL-20, (Okawara Kakoki Co.), may be used for the spray drying method using rotary-disk atomizer. The nozzle of the atomizer that produces the powder of the present application may include, for example, nozzle types 1A, 1, 2A, 2, 3 (Yamato Chemical Co.) or similar commercially available nozzles, may be used for the above-mentioned spray drier. In addition, disks type MC-50, MC-65 or MC-85 (Okawara Kakoki Co.) may be used as rotary disks of the spray-drier atomizer.

In one aspect, the spray drying devices traditionally used for the industrial manufacture of a milk or coffee powder may also be employed in the present method. See Jensen J. D., Food Technology, June, 60-71, 1975. In one aspect, the spray drying devices may include those described in U.S. Pat. No. 4,702,799 (Nestle). In one embodiment, operation of the spray drier may be performed at about 200-400° C. at the end of the spray nozzle where the rest of the device may be operated at a lower temperature which may reach the air outlet temperature, such as the sprayer described in U.S. Pat. No. 3,065,076 (Nestle).

In another aspect, the spray-drying apparatus used in the process of the present application may be any of the various commercially available apparati. Representative examples of spray drying apparati are the Anhydro Dryers (Anhydro Corp., Attleboro Falls, Mass.), the Niro Dryer (Niro Atomizer Ltd., Copenhagen, Denmark) or a Leaflash apparatus (CCM Sulzer). In one aspect, a spray-drier with a pressure nozzle may be used.

In another aspect, the powder obtained from the drying process may comprise 10% by weight, 20% by weight, 30% by weight, 40% by weight, 50% by weight, 60% by weight, 70% by weight, 80% by weight, or 90% by weight or more of particles having an average particle size in the range from about 5 to 1,000 microns, from about 10 to 500 microns, from about 10 to 350 microns, from about 20 to 250 microns, or about 40 to 200 microns, or about 50 to 150 microns.

The dry composition of the present application may be formulated to provide a dry powder that is stable, and may form a partially turbid solution, a milky or cloudy solution, or a clear solution as desired. Where a substantially clear solution or composition is not desired, such as a milky or cloudy solution or composition is desired as obtained from the dry powder, the ratio of the solubilizing agent, such as TPGS, Solutol HS 15 or Cremophor EL, to the POFA may be reduced. For example, the ratio (wt/wt) of TPGS, Solutol HS 15, or Cremophor EL to POFA (e.g., TPGS:POFA) may be reduced to a range of about 2:1 to about 1.5:1, about 1.3:1, about 1:1, or 0.9:1 or less.

The dry powder formulation of the present application provides POFA or hill oil compositions that are stable to decomposition. Without being bound by any theory presented herein, it is believed that the judicious selection of the solid support allows the encapsulation of the POFA, provides substantially no surface oil and shields the POFA from oxidation by exposure to ambient air. In addition, the dry powder formulation is readily re-dissolved in water and forms a clear solution.

The concentrated powder may be prepared as dry preparations, such as, for example, a powder, a granular material, a crystalline material and other types of dry particle preparations or combinations thereof. In one aspect, the dry preparations may be prepared by mixing the ingredients and compositions, as disclosed herein, to form a concentrated solution, and then drying the solution to a dry powder form by conventional drying methods. Representative drying methods may include, for example, lyophilization (or freeze drying), spray drying, fluid bed drying, drum drying, pulse combustion drying and various combinations thereof. In one aspect of the drying method, the method is a spray drying method.

### Surfactants or Solubilizing Agents:

One or more surfactants (or solubilizing agents), or a mixture of surfactants may be used in the present formulations. Representative surfactants employed may include: HLB≥10 surfactants such as Poloxamer 188, Polysorbate 80, Polysorbate 20, Vit E-TPGS, Solutol HS 15, PEG-40 Hydrogenated castor oil (Cremophor RH40), PEG-35 Castor oil (Cremophor EL), PEG-8-glyceryl capylate/caprate (Labrasol), PEG-32-glyceryl laurate (Gelucire 44/14), PEG-32-glyceryl palmitostearate (Gelucire 50/13); HLB 8-12 such as Polysorbate 85, Polyglyceryl-6-dioleate (Caprol MPGO), Mixtures of high and low HLB emulsifiers; and LB≤8 such as Sorbitan monooleate (Span 80), Capmul MCM, Maisine 35-1, Glyceryl monooleate, Glyceryl monolinoleate, PEG-6-glyceryl oleate (Labrafil M 1944 CS), PEG-6-glyceryl linoleate (Labrafil M 2125 CS), Oleic acid, Linoleic acid, Propylene glycol monocaprylate (e.g. Capmul PG-8 or Capryol 90), Propylene glycol monolaurate (e.g., Capmul PG-12 or Lauroglycol 90), Polyglyceryl-3 dioleate (Plurol Oleique CC497), Polyglyceryl-3 diisostearate (Plurol Diisostearique) and Lecithin with and without bile salts.

### Batch Process for Preparing Stabilized TPGS and POFA Composition:

Generally, the process for preparing stabilized TPGS/POFA compositions (or Solutol HS 15 or Cremophor EL/POFA compositions) may include heating the TPGS at an elevated temperature sufficient to melt the TPGS. The mixture may be performed in an inert atmosphere, such as under nitrogen. A mixture of water, di-sodium EDTA or calcium disodium EDTA, ascorbic acid, vitamin C palmitate and sodium bisulfite is added to the TPGS. In one embodiment, the water is heated to about 50° C. before the addition of di-sodium EDTA or calcium disodium EDTA, ascorbic acid, vitamin C palmitate, sodium bisulfite and an antioxidant such as alpha tocopherol or mixture of alpha, beta, gamma and delta forms of tocopherols, or a blend of a mixture of tocopherols that is high in delta tocopherol, Fortium MTD10 (MTD10, Kemin Food Technologies), or a water soluble antioxidants, may be heated to above 45° C., or about 45° C. to 55° C. and then added to the combined mixture.

In another embodiment, a vessel containing water is heated to about 50° C., and a mixture of di-sodium EDTA or calcium disodium EDTA, ascorbic acid, vitamin C palmitate and sodium bisulfite is added to the vessel and heated to about 45° C. to about 55° C. In certain aspects of the process, sodium metabisulfite, potassium bisulfite, or potassium metabisulfite may be used in place of sodium bisulfite. Fortium MTD10 is preheated above 45° C., or about 45° C. to 55° C. and then added to the combined mixture. TPGS may be preheated to about 45° C. to about 55° C. and then added to a vessel.

The resulting mixture, prepared in the embodiment described above, may be heated and stirred at an elevated temperature for a sufficient period of time to allow complete mixing. The mixture may be heated at about 45° C. to about 98° C., or about 55° C. to 98° C., about 85° C. to 98° C., about 90° C. to 98° C., or about 95° C. to 97° C. In one embodiment, the mixture is heat above 95° C. for a sufficient period of time to provide a homogeneous slurry. At the present state of the composition that is described as a “homogeneous slurry” (or solution) means that the slurry composition comprising the various elements or additives are sufficiently well mixed. Depending on the batch size, the heated mixture may be heated at the desired temperature for at least 10 minutes, at least 15 minutes, at least 30 minutes, at least 45 minutes or at least about 60 minutes to attain a homogeneous solution. The resulting stirred slurry is cooled at a rate of about 5° C. to 20° C. per hour, 5° C. to 15° C. per hour, or about 10° C. per hour until the mixture reaches about 25° C. or at ambient temperature.

A.1. Into a 500 liters vessel is added purified water (119 kg). The vessel is heated to about 50° C. under nitrogen, and the solution is agitated for about 5 minutes. To the vessel is added di-Na EDTA (2.05 kg), ascorbic acid (3.41 kg), vitamin C palmitate (ascorbyl palmitate, 2.56 kg) and sodium bisulfite (0.733 kg). Fortium MTD10 (2.56 kg) is preheated in a separate vessel to about 45-55° C., and added to the 500 liters vessel. The resulting vessel is stirred and heated to about 95-97° C. for about 15 minutes. TPGS (34.1 kg) is preheated in a separate vessel to 45-55° C., and added to the 500 liters vessel. The resulting mixture is stirred for about 15 minutes until the solution is homogeneous. POFA (17.0 kg) is added to the vessel, and the mixture is heated to about 95-97° C. for about 30 minutes. A 4 oz sample is obtained, allowed to cool to about 25° C. and tested for solution homogeneity. The mixture in the vessel is stirred until the solution is homogeneous.

As provided herein, the POFA compositions that are typically employed may have a purity range of about 70-85%, 80-85% and 85-90%. However, higher purity or lower purity ranges may also be employed.

The resulting stirred mixture is cooled at a rate of about 10° C. per hour until the mixture is cooled to about 25° C. The solution is stirred at 25° C. for 5 minutes. The resulting solution is transferred and stored in a shipping container under nitrogen. In one embodiment, the aqueous solution is prepared under conditions that are suited for human consumption and is further treated for the inactivation of microbes by a process selected from the group consisting of pasteurization, aseptic packaging, membrane permeation, sonication or combinations thereof.

A.1.3. Into a 500 liters vessel is added purified water (119 kg). The vessel is heated to about 50° C. under nitrogen, and the solution is agitated for about 5 minutes. To the vessel is added calcium disodium EDTA (2.05 kg), ascorbic acid (3.41 kg), vitamin C palmitate (ascorbyl palmitate, 2.56 kg) and sodium bisulfite (0.0733 kg). Fortium MTD10 (2.56 kg) is preheated in a separate vessel to about 45-55° C., and added to the 500 liters vessel. The resulting vessel is stirred and heated to about 95-97° C. for about 15 minutes. TPGS (34.1 kg) is preheated in a separate vessel to 45-55° C., and added to the 500 liters vessel. The resulting mixture is stirred for about 15 minutes until the solution is homogeneous. POFA (17.0 kg) is added to the vessel, and the mixture is heated to about 95-97° C. for about 30 minutes. The resulting stirred mixture is cooled at a rate of about 10° C. per hour until the mixture is cooled to about 25° C.

A.2. Into a 22 liter round bottom flask under a blanket of nitrogen is added water (5.91 kg). To the stirred water is added ascorbic acid (0.170 kg), ethylenediaminetetraacetic acid disodium salt dihydrate (Di-Na EDTA, 0.101 kg), Fortium MTD10 (0.127 kg), L-ascorbic acid-6-palmitate (0.127 kg) and sodium bisulfite (0.0036 kg). The resulting mixture is stirred, heated to 90-95° C. for about 55 minutes. TPGS (1.69 kg) is heated to about 50° C. and then added to the mixture. The resulting solution is stirred at 90-95° C. for about 30 minutes. High grade POFA (0.844 kg) is added to the flask by cannula under nitrogen pressure, and the resulting mixture is stirred at 96-98° C. for about 30 minutes. The mixture is cooled from about 97° C. to about 31° C. in about 1 hour.

A.1.5 Into a 500 liters vessel is added purified water (119 kg). The vessel is heated to about 50° C. under nitrogen, and the solution is agitated for about 5 minutes. To the vessel is added Di-Na EDTA (2.05 kg), ascorbic acid (3.41 kg), vitamin C palmitate (ascorbyl palmitate, 2.56 kg) and sodium bisulfite (0.0733 kg). Alpha-D-tocopherol (2.56 kg) is preheated in a separate vessel to about 45-55° C., and added to the 500 liters vessel. The resulting vessel is stirred and heated to about 95-97° C. for about 15 minutes. TPGS (34.1 kg) is preheated in a separate vessel to 45-55° C., and added to the 500 liters vessel. The resulting mixture is stirred for about 15 minutes until the solution is homogeneous. POFA (17.0 kg) is added to the vessel, and the mixture is heated to about 95-97° C. for about 30 minutes. The solution is stirred at 25° C. for 5 minutes.

A.3.7 Into a 22 liter round bottom flask under a blanket of nitrogen is added water (5.91 kg). To the stirred water is added ascorbic acid (0.170 kg), ethylenediaminetetraacetic acid calcium disodium salt (Calcium Disodium EDTA, 0.101 kg), Fortium MTD10 (0.127 kg), L-ascorbic acid-6-palmitate (0.127 kg) and sodium bisulfite (0.0036 kg). The resulting mixture is stirred, heated to 90-95° C. for about 55 minutes. TPGS (1.69 kg) is heated to about 50° C. and then added to the mixture. The resulting solution is stirred at 90-95° C. for about 30 minutes. High grade POFA (0.844 kg) is added to the flask by cannula under nitrogen pressure, and the resulting mixture is stirred at 96-98° C. for about 30 minutes. The mixture is cooled from about 97° C. to about 31° C. in about 1 hour.

In one embodiment, the clear aqueous solution is prepared under conditions that are suited for human consumption and is further treated for the inactivation of microbes by a process selected from the group consisting of pasteurization, aseptic packaging, membrane permeation, sonication or combinations thereof.

TABLE 1

| | Experiments | | | | | | | |
|---|---|---|---|---|---|---|---|---|
| Reagents (kg) | A.1.1 | A.1.2 | A.1.3 | A.1.4 | A.2.1 | A.2.2 | A.2.3 | A.2.4 |
| Water (purified) | 89.462 | 149.103 | 89.462 | 149.103 | 4.433 | 7.388 | 4.433 | 7.388 |
| Di-Na EDTA | 1.534 | 2.556 | 1.534 | 2.556 | 0.0758 | 0.1263 | 0.0758 | 0.1263 |
| Ascorbic acid | 2.556 | 4.260 | 2.556 | 4.260 | 0.128 | 0.213 | 0.128 | 0.213 |
| Vitamin C palmitate | 1.917 | 3.195 | 1.917 | 3.195 | 0.0953 | 0.159 | 0.0953 | 0.159 |
| (ascorbyl palmitate) | | | | | | | | |
| Sodium bisulfite | 0.0550 | 0.0916 | 0.550 | | 0.027 | 0.045 | 0.27 | |
| Potassium bisulfite | | | | 0.0916 | | | | 0.045 |
| Fortium MTD10^{a} | 1.917 | 3.195 | 1.917 | 3.195 | 0.0953 | 0.159 | 0.0953 | 0.159 |
| TPGS^{b} | 25.50 | 42.60 | 25.50 | 42.60 | 1.271 | 2.118 | 1.271 | 2.118 |
| POFA | 10.53 | 17.55 | 10.53 | 17.55 | 0.633 | 1.055 | 0.633 | 1.055 |

^{a}In other experiments using the same ratio of reagents and additives, Fortium MTD10 may be replaced with synthetic or natural tocopherol, alpha-D-tocopherol, or a mixture of natural tocopherols.
^{b}In other experiments, TPGS is replaced with Solutol HS 15 or Cremophor EL.

TABLE 2

| | Experiments | | |
|---|---|---|---|
| | Relative Wt/Wt % Ranges of Reagents | | |
| Reagents | A.3.1 | A.3.2 | A.3.3 |
| High Grade POFA | 6.0 to 14 | 5.0 to 15 | 3.0 to 20 |
| TPGS | 13 to 25 | 11 to 27 | 10 to 30 |
| Water | 47 to 88 | 45 to 95 | 40 to 97 |
| Ascorbic acid | 0.01 to 0.5 | 0.001 to 1.0 | 0.001 to 2.0 |
| Disodium EDTA | 0.50 to 2.0 | 0.01 to 2.5 | 0.005 to 5.0 |
| MTD-10 | 0.5 to 3.0 | 0.01 to 5.0 | 0.005 to 10.0 |
| Ascorbic Acid 6- | 0.5 to 3.0 | 0.01 to 5.0 | 0.005 to 10.0 |
| Palmitate | | | |
| Sodium bisulfite | 0.01 to 0.1 | 0.001 to 0.5 | 0.001 to 1.0 |

TABLE 3

| | Experiments | | |
|---|---|---|---|
| | Relative Wt/Wt % Ranges of Reagents | | |
| Reagents | A.3.4 | A.3.5 | A.3.6 |
| High Grade POFA | 6.0 to 14 | 5.0 to 15 | 3.0 to 20 |
| TPGS | 13 to 25 | 11 to 27 | 10 to 30 |
| Water | 47 to 88 | 45 to 95 | 40 to 97 |
| Ascorbic acid | 0.01 to 0.5 | 0.001 to 1.0 | 0.001 to 2.0 |
| Calcium Disodium | 0.50 to 2.0 | 0.01 to 2.5 | 0.005 to 5.0 |
| EDTA | | | |
| MTD-10 | 0.5 to 3.0 | 0.01 to 5.0 | 0.005 to 10.0 |
| Ascorbic Acid 6- | 0.5 to 3.0 | 0.01 to 5.0 | 0.005 to 10.0 |
| Palmitate | | | |
| Sodium bisulfite | 0.01 to 0.1 | 0.001 to 0.5 | 0.001 to 1.0 |

Qualitative analysis of the products obtained from the process described herein shows that the product meets all specifications established for fatty acid composition, physical properties, trace impurities and microbials content.

### Procedure for Preparing Stabilized Surfactant-POFA Emulsions for Spray Drying:

Generally, the process for preparing stabilized TPGS/POFA emulsions include the addition of one or more additives and/or carriers, such as a starch or a polymer, to water, and the resulting mixture is heated above room temperature. The mixture may be heated to about 35° C. to 90° C., about 35° C. to about 80° C., about 35° C. to 75° C., or about 50° C. to 70° C., about 60° C. to 70° C. or about 65° C. Depending on the nature of the additives and the size of the batch, the mixture may be heated from at least about 5 minutes, at least 10 minutes, at least 15 minutes, at least 30 minutes, at least 45 minutes or at least about 60 minutes to about 120 minutes. The resulting mixture is then cooled to below room temperature, about 15° C. to 20° C., about 5° C. to 15° C., or about 10° C. To the stirred mixture is then added TPGS/POFA/stabilized composition, and the resulting emulsion is stirred for at least about 5 minutes, about 10 minutes, about 15 minutes, about 30 minutes or at least about 60 minutes to provide the predrying emulsion. The predrying emulsion may be used in the subsequent drying step as disclosed herein.

As provided herein, the additives and/or carriers may include HI-CAP 100 (National Starch), Emcap Starch, TICAMULSION FC (TIC GUMS), Spray gum F (gum acacia with Maltrin-100), natural vanillin, natural maltol, maltodextrin 10-DE, and other additives as disclosed herein and mixtures thereof. In one embodiment, the carrier is maltodextrin and Spray gum F. In one embodiment, the ratio (wt/wt) of water to TPGS/03-EE/stabilized ranges from about 0.3:1 to 10:1, about 0.5:1 to about 5:1, about 0.5:1 to about 3:1, about 1:1 to about 2.5:1, and about 1.5:1 to about 2:1. In one embodiment, the ratio (wt/wt) of the additives and/or carriers to the TPGS/POFA/stabilized composition may range from about 0.1:1 to about 100:1, about 0.1:1 to 50:1; or about 0.3:1 to 30:1, about 0.5:1 to 15:1, or about 0.3:1 to about 10:1.

A.4. Into a vessel equipped with an overhead stirrer as added water (543.5 g), and the water solution is stirred at room temperature. To the vigorously stirred solution is added HI-CAP 100 (National Starch, 109 g), and the mixture is stirred for 15 minutes. The resulting mixture is heated to 65.5° C. and mixed for 5 minutes. The mixture is cooled to 10° C. with mixing. TPGS/POFA/stabilized emulsion (“OTECH emulsion,” 348 g) is added and the resulting mixture is stirred for 5 minutes to provide the pre-drying emulsion.

A.5. Into a vessel equipped with an overhead stirrer as added water (1,430 g), and the water solution is stirred at room temperature. To the vigorously stirred solution is added Emcap Starch (Cargill, 648 g), and the mixture is stirred for 15 minutes. The resulting mixture is heated to 65.5° C. and mixed for 5 minutes. The mixture is cooled to 10° C. with mixing. TPGS/POFA/stabilized emulsion (918 g) is added and the resulting mixture is stirred for 5 minutes to provide the pre-drying emulsion.

A.6. Into a vessel equipped with an overhead stirrer as added water (468.7 g), and the water solution is stirred at room temperature. To the vigorously stirred solution is added Emcap Starch (Cargill, 281.3 g), and the mixture is stirred for 15 minutes. The resulting mixture is heated to 65.5° C. and mixed for 5 minutes. The mixture is cooled to 10° C. with mixing. TPGS/POFA/stabilized emulsion (250.0 g) is added and the resulting mixture is stirred for 5 minutes to provide the pre-drying emulsion.

A.7. Into a vessel equipped with an overhead stirrer is added water (500 g), and the water solution is stirred at room temperature. To the vigorously stirred solution is added TICAMULSION FC (TIC GUMS, 180.0 g), and the mixture is stirred for 15 minutes. The resulting mixture is heated to 65.5° C. and mixed for 5 minutes. The mixture is cooled to 10° C. with mixing. TPGS/POFA/stabilized emulsion (320 g) is added and the resulting mixture is stirred for 5 minutes to provide the pre-drying emulsion.

A.8. Into a vessel equipped with an overhead stirrer is added water (531.9 g), and the water solution is stirred at room temperature. To the vigorously stirred solution is added TICAMULSION FC (TIC GUMS, 255.3 g), and the mixture is stirred for 15 minutes. The resulting mixture is heated to 65.5° C. and mixed for 5 minutes. The mixture is cooled to 10° C. with mixing. TPGS/POFA/stabilized emulsion (212.8 g) is added and the resulting mixture is stirred for 5 minutes to provide the pre-drying emulsion.

A.9. Into a vessel equipped with an overhead stirrer is added water (425.0 g), and the water solution is stirred and heated to about 18° C. to 24° C. To the vigorously stirred solution is added Spray gum F (gum acacia with Maltrin-100, 85 g), natural vanillin (0.85 g), natural maltol (0.21 g) and maltodextrin 10-DE (212.5 g), and the mixture is stirred for about 15 minutes. The resulting mixture is heated to about 63° C. to 68° C. and mixed for 5 to 10 minutes. The mixture is cooled to about 7.2° C. to 12.8° C. with mixing. TPGS/POFA/stabilized emulsion (425 g) is added and the resulting mixture is stirred for 5 minutes to provide the predrying emulsion.

A.10. Into a vessel equipped with an overhead stirrer is added water (425.0 g), and the water solution is stirred and heated to about 18° C. to 24° C. To the vigorously stirred solution is added Spray gum F (gum acacia with Maltrin-100, 85 g), natural maltol (0.21 g) and maltodextrin 10-DE (212.5 g), and the mixture is stirred for about 15 minutes. The resulting mixture is heated to about 63° C. to 68° C. and mixed for 5 to 10 minutes. The mixture is cooled to about 7.2° C. to 12.8° C. with mixing. TPGS/POFA/stabilized emulsion (425 g) is added and the resulting mixture is stirred for 5 minutes to provide the predrying emulsion.

A.11. Into a vessel equipped with an overhead stirrer is added water (425 g), and the water solution is stirred and heated to about 18° C. to 24° C. To the vigorously stirred solution is added Spray gum F (gum acacia with Maltrin-100, 85 g), natural vanillin (0.85 g) and maltodextrin 10-DE (213 g), and the mixture is stirred for about 15 minutes. The resulting mixture is heated to about 63° C. to 68° C. and mixed for 5 to 10 minutes. The mixture is cooled to about 7.2° C. to 12.8° C. with mixing. TPGS/POFA/stabilized emulsion (425 g) is added and the resulting mixture is stirred for 5 minutes to provide the pre-drying emulsion.

A.12. Into a vessel equipped with an overhead stirrer is added water (425.0 g), and the water solution is stirred and heated to about 18° C. to 24° C. To the vigorously stirred solution is added natural vanillin (0.85 g), natural maltol (0.21 g) and maltodextrin 10-DE (298 g), and the mixture is stirred for about 15 minutes. The resulting mixture is heated to about 63° C. to 68° C. and mixed for 5 to 10 minutes. The mixture is cooled to about 7.2° C. to 12.8° C. with mixing. TPGS/POFA/stabilized emulsion (425 g) is added and the resulting mixture is stirred for 5 minutes to provide the pre-drying emulsion.

TABLE 4

| | Examples | | | | | | | |
|---|---|---|---|---|---|---|---|---|
| Reagents (grams) | A.9 | A.9.1 | A.10.1 | A.10.2 | A.11.1 | A.11.2 | A.12.1 | A.12.2 |
| Water | 425 | 575 | 425 | 575 | 425 | 575 | 425 | 575 |
| Spray gum F (gum acacia | 85 | 115 | 85 | 115 | 85 | 115 | | |
| with Maltrin-100) | | | | | | | | |
| Natural vanillin | 0.85 | 1.15 | | | 0.85 | 1.15 | 0.85 | 1.15 |
| Natural maltol | 0.21 | 0.29 | 0.21 | 0.29 | | | 0.21 | 0.29 |
| Maltodextrin 10-DE | 212.5 | 287.5 | 212.5 | 287.5 | 212.5 | 287.5 | 297.5 | 402.5 |
| TPGS/POFA/stabilized | 425 | 575 | 425 | 575 | 425 | 575 | 425 | 575 |

The emulsions prepared according to the above procedure may be dried using various drying methods as provided herein. In one embodiment, the emulsions may be dried using the spray drying methods as described herein. The spray dried composition comprises water content from about 1% to about 10%, from about 1% to about 6%, about 2% to about 5%, about 3% to 4%, about 1% to 3%, about 2% to 3%, about 3% to 6%, about 3% to 5%, or about 3% to 4%. Accordingly, the clarity or homogeneity of the aqueous solution containing the compositions as described herein may be controlled by the amount of residual water remaining in the dried powders.

### Method for Making a POFA Beverage:

In another aspect, the present application provides a method for making a beverage (e.g., a non-alcoholic beverage) that includes omega fatty acids. An exemplary method includes: contacting an original beverage with a water-soluble POFA stock solution (e.g., POFA-50 stock solution) of the present application. Exemplary original beverages are disclosed herein and include carbonated or uncarbonated water, flavored water, soft drinks, beer and drinkable dairy products. In one example, the method further includes adding a vitamin (e.g., vitamin C, vitamin E, a B-vitamin (e.g., vitamin B-pentapalmitate) or combinations thereof) to the beverage. In one example, when the vitamin (e.g., vitamin E) is added to the beverage, the vitamin is first solubilized in an aqueous medium using a solubilizing agent, such as a solubilizing agent of the present application, and is subsequently added to the beverage. Exemplary solubilizing agents that can be used to solubilize the vitamin (e.g., vitamin E) include TWEEN-85, TPGS, TPGS-1000 and polyoxyethylene sorbitan monooleate, and solubilizing agents as disclosed herein. In another embodiment, the present application provides a beverage produced by any of the above methods of the present application. In yet another example according to any of the above embodiments, the POFA comprises a compound selected from omega-3-fatty acids, omega-6-fatty acid, carotenoids, essential oils, flavor oils and lipophilic vitamins, and mixtures thereof. In one example, the omega-3-fatty acid is selected from docosahexaenoic acid (DHA), eicosapentaenoic acid (EPA) and alpha-linolenic acid (ALA).

### Methods and Procedures:

As provided herein, the present application provides a method for preparing clear and stable POFA compositions for use in various food products. The compositions are ideally GRAS (or FDA-GRAS self-affirmed GRAS (TPGS-1000)), or the composition comprises other food materials.

In one aspect, the ratio of the surfactant to the POFA is low, such as a ratio of less than 2:1, less than 1:1 (w/w), less than 0.75:1 (w/w) or less than 0.5:1 (w/w).

In certain embodiments, the formulations comprise a high percentage of the daily allowable dose of the emulsion ingredient such that omega-3 fatty acids are provided in high delivery dosages. In a particular aspect, the emulsifier that is present does not present a significant taste and odor profile. In certain embodiments, the surfactants employed in the present application may include:

Hydrophilic Lipophilic Balance is as defined in the art as HLB=20*Mh/M, where Mh is the molecular mass of the hydrophilic portion of the Molecule, and M is the molecular mass of the whole molecule, giving a result on an arbitrary scale of 0 to 20. An HLB value of 0 corresponds to a completely hydrophobic molecule, and a value of 20 would correspond to a molecule made up completely of hydrophilic components. The HLB value can be used to predict the surfactant properties of a molecule. For example, a value from 0 to 3 indicates an anti-foaming agent; a value from 4 to 6 indicates a W/O (water in oil) emulsifier; a value from 7 to 9 indicates a wetting agent; a value from 8 to 18 indicates an O/W (oil in water) emulsifier; a value from 13 to 15 is typical of detergents; a value of 10 to 18 indicates a solubiliser or hydrotrope. HLB>10 may include Poloxamer 188, Polysorbate 80, Polysorbate 20, Vitamin E-TPGS, Solutol HS 15, PEG-40, Hydrogenated castor oil (Cremophor RH40), PEG-35 Castor oil (Cremophor EL), PEG-8-glyceryl capylate/caprate (Labrasol), PEG-32-glyceryl laurate (Gelucire 44/14), PEG-32-glyceryl palmitostearate (Gelucire 50/13). HLB 8-12 may include Polysorbate 85, polyglyceryl-6-dioleate (Caprol MPGO), TPGS, and/or mixtures of high and low HLB emulsifiers. HLB<8 may include sorbitan monooleate (Span 80), Capmul MCM, maisine 35-1, glyceryl monooleate, glyceryl monolinoleate, PEG-6-glyceryl oleate (Labrafil M 1944 CS), PEG-6-glyceryl linoleate (Labrafil M 2125 CS), oleic acid, linoleic acid, propylene glycol monocaprylate (e.g. Capmul PG-8 or Capryol 90), propylene glycol monolaurate (e.g., Capmul PG-12 or lauroglycol 90), polyglyceryl-3 dioleate (Plurol Oleique CC497), polyglyceryl-3 diisostearate (Plurol Diisostearique) and lecithin with and without bile salts.

The relative solubility of compositions of the present application, including composition comprising, for example, a 2:1 and 1:1 surfactant/POFA systems in water (or other aqueous solvent system(s)) may be determined by emulsification screening, visual appearance, turbidity, tarticle (emulsion droplet) size by Photon Correlation Spectroscopy (PCS), visual assessment of dilution effects, ambient room temperature (RT) stability at 1, 2 and 4 weeks and established compatibility with beverage matrices.

As provided herein, the compositions of the present application demonstrate significant oxidative stability, and may be tested and determined by storing the composition in vials. The composition may be purged with oxygen and analyzed at various time intervals to determine compositions having the optimal appearance, the assay (by HPLC, for example), by PCS and the physical and chemical stability suitable for use in various food products.

### Examples

Solubilization of Phospholipid Comprising Krill Oils (POFA) with TPGS:

In a microcentrifuge tube, Omega-3 Food Grade Krill Oil (100 mg, Ocean Nutrition Canada Ltd.), Vitamin E TPGS (200 mg, Antares) and DI water (700 mg) are combined. The mixture is heated to 90-100° C. until it became homogeneous. The homogeneous mixture is cooled to room temperature and is an opaque homogeneous mixture. Then 60 mg of the opaque homogeneous mixture is diluted with 30 mL of DI water. Sample of the composition is then filtered through a 0.2 micron filter.

In a 250 mL 3-neck round bottom flask equipped with an overhead stirrer, thermocouple, condenser, heating mantle, addition funnel and a nitrogen inlet, Omega 3 Food Grade Krill Oil (11.4 g, Ocean Nutrition Canada Ltd.), Vitamin E TPGS (22.8 g, Antares) is added and heated to 90° C. until melted. DI Water (70 g, 90° C.) is added via cannula in one portion. After the addition is complete, the mixture is heated to 90° C. until it became homogeneous. The homogeneous mixture (65.6 mg) is diluted with DI water (30 mL). Preparation of the Stabilized Emulsion of TPGS and High Grade Krill Oil:

In a 250 mL 3-neck round bottom flask equipped with an overhead stirrer, thermocouple, condenser, heating mantle and a nitrogen inlet, Vitamin E TPGS (20.0 g, TR Nutritionals), Vitamin C (0.15 g, Sigma), EDTA disodium (0.4 g, Sigma), Vitamin C Palmitate (0.6 g, Alfa Aesar), Vitamin E (0.6 g, Kemin), high grade hill oil (10.0 g, Organic Technologies) and DI water (70 g) are combined. The mixture is heated to 95° C. until it became homogeneous, and is held for ˜45 minutes. Then the opaque homogeneous mixture is cooled in an ice water bath. After cooling to 5° C., the mixture remained homogeneous but is opaque.

Preparation of the Stabilized Emulsion of TPGS and High Grade Phospholipid Comprising Krill Oil, with Additional Bisulfite:

In a 250 mL 3-neck round bottom flask equipped with an overhead stirrer, thermocouple, condenser, heating mantle and a nitrogen inlet, Vitamin E TPGS (20.0 g, Antares), Vitamin C (0.2 g, Sigma), EDTA disodium (0.4 g, Sigma), Vitamin C Palmitate (0.5 g, Alfa Aesar), sodium metabisulfite (0.5 g, Sigma-Aldrich), Vitamin E (0.5 g, Kemin), high grade hill oil (10.0 g, Organic Technologies) and DI water (70 g) are combined. The mixture is heated to 95° C. until it became homogeneous, and is held for 50 minutes. Then the opaque homogeneous mixture is cooled in an ice water bath. At 63° C. the mixture became a clear homogeneous mixture.

### Formulation Using High Grade Krill Oil:

TPGS-1000 (20 g, Antares), high grade hill oil (10 g, Organic Technologies) and water (70 g) are charged to a 250 mL 3-neck RBF. The mixture is heated to 92.8° C., where upon a thick homogeneous mixture is observed. This is held at 92.8 to 95° C. for ˜30 minutes, after which is cooled in an ice bath. At 85° C. a clear light yellow solution is observed, but is cooled to 2.8° C. This is then reheated and cooled to a clear solution, and a small sample is taken and placed in the refrigerator to monitor stability further. After a day in a vial, there is little to no detectable odor.

Solubilization of Phospholipid Comprising Krill Oils (POFA) with Solutol HS 15 or Cremophor EL:

In a microcentrifuge tube, Omega-3 Food Grade Krill Oil (100 mg, Ocean Nutrition Canada Ltd.), Solutol HS 15 or Cremophor EL (200 mg) and DI water (700 mg) are combined. The mixture is heated to 90-100° C. until it became homogeneous. The homogeneous mixture is cooled to room temperature and is an opaque homogeneous mixture. Then 60 mg of the opaque homogeneous mixture is diluted with 30 mL of DI water. Sample of the composition is then filtered through a 0.2 micron filter.

In a 250 mL 3-neck round bottom flask equipped with an overhead stirrer, thermocouple, condenser, heating mantle, addition funnel and a nitrogen inlet, Omega 3 Food Grade Krill Oil (11.4 g, Ocean Nutrition Canada Ltd.), Solutol HS 15 or Cremophor EL (20 g) is added and heated to 90° C. until melted. DI Water (70 g, 90° C.) is added via cannula in one portion. After the addition is complete, the mixture is heated to 90° C. until it became homogeneous. The homogeneous mixture (65.6 mg) is diluted with DI water (30 mL).

### Preparation of the Stabilized Emulsion of Solutol HS 15 or Cremophor EL and High Grade Krill Oil:

In a 250 mL 3-neck round bottom flask equipped with an overhead stirrer, thermocouple, condenser, heating mantle and a nitrogen inlet, Solutol HS 15 or Cremophor EL (20.0 g), Vitamin C (0.15 g, Sigma), EDTA disodium (0.4 g, Sigma), Vitamin C Palmitate (0.6 g, Alfa Aesar), Vitamin E (0.6 g, Kemin), high grade hill oil (10.0 g, Organic Technologies) and DI water (70 g) are combined. The mixture is heated to 95° C. until it became homogeneous, and is held for ˜45 minutes. Then the opaque homogeneous mixture is cooled in an ice water bath. After cooling to 5° C., the mixture remained homogeneous but is opaque.

Preparation of the Stabilized Emulsion of Solutol HS 15 or Cremophor EL and High Grade Phospholipid Comprising Krill Oil, with Additional Bisulfite:

In a 250 mL 3-neck round bottom flask equipped with an overhead stirrer, thermocouple, condenser, heating mantle and a nitrogen inlet, Solutol HS 15 or Cremophor EL (20.0 g), Vitamin C (0.2 g, Sigma), EDTA disodium (0.4 g, Sigma), Vitamin C Palmitate (0.5 g, Alfa Aesar), sodium metabisulfite (0.5 g, Sigma-Aldrich), Vitamin E (0.5 g, Kemin), high grade hill oil (10.0 g, Organic Technologies) and DI water (70 g) are combined. The mixture is heated to 95° C. until it became homogeneous, and is held for 50 minutes. Then the opaque homogeneous mixture is cooled in an ice water bath. At 63° C. the mixture became a clear homogeneous mixture.

### Formulation Using High Grade Krill Oil:

Solutol HS 15 or Cremophor EL (20 g), high grade hill oil (10 g, Organic Technologies) and water (70 g) are charged to a 250 mL 3-neck RBF. The mixture is heated to 92.8° C., where upon a thick homogeneous mixture is observed. This is held at 92.8 to 95° C. for ˜30 minutes, after which is cooled in an ice bath. At 85° C. a clear light yellow solution is observed, but is cooled to 2.8° C. This is then reheated and cooled to a clear solution, and a small sample is taken and placed in the refrigerator to monitor stability further. After a day in a vial, there is little to no detectable odor.

While a number of exemplary embodiments, aspects and variations have been provided herein, those of skill in the art will recognize certain modifications, permutations, additions and combinations and certain sub-combinations of the embodiments, aspects and variations. It is intended that the following claims are interpreted to include all such modifications, permutations, additions and combinations and certain sub-combinations of the embodiments, aspects and variations are within their scope.

What is claimed is:

1. A stable, water soluble formulation comprising:
a) a phospholipid comprising omega fatty acids; and
b) one or more additives selected from the group consisting of a metal chelator, a water soluble reducing agent, a lipophilic reducing agent, a bisulfite salt, a metabisulfite salt or mixtures thereof, wherein the formulation further comprises a solubilizing agent selected from Solutol HS 15, Cremophor EL, TPGS (polyoxyethanyl-a-tocopheryl succinate) or TPGS-1000 (D-alpha-tocopheryl polyethylene glycol 1000 succinate), wherein the tocopheryl is the natural tocopherol isomer or the un-natural tocopherol isomer.

2. (canceled)

3. The formulation of claim 1, wherein the phospholipid comprising omega fatty acids is a natural or reconstituted phospholipid comprising omega fatty acids.

4. The formulation of claim 3, wherein the natural or reconstituted phospholipid comprising omega fatty acids comprise:
a) omega fatty acids and their mono- and diphospholipid isomers, and combinations thereof;
b) omega fatty acid ethyl esters selected from the group consisting of the C₁-C₁₀ alkyl esters, the C₁-C₅ alkyl esters, the C₁-C₃ alkyl esters or the C₂-C₅ alkyl esters, and mixtures thereof;
c) a monophosphate ester derivative selected from the group consisting of 1-, 2- or 3-isomer or mixtures thereof; a diphosphate derivative selected from the group consisting of the 1,2- or 1,3-isomer or mixtures thereof;
d) a mixture of mono- and diphosphate derivatives and their isomers; or
e) mixtures of a), b), c) and d) thereof;
wherein the omega fatty acid is an omega-3 fatty acid, omega-6 fatty acid, omega-9 fatty acid, omega-12 fatty acid, and mixtures thereof.

5. (canceled)

6. The formulation of claim 1, wherein the phospholipids comprising omega fatty acids comprise a compound selected from the group consisting of omega-3 fatty acid, omega-6 fatty acid, omega-9 fatty acid, omega-12 fatty acid, the phospholipid esters of the omega fatty acids, the glyceride esters of the omega fatty acids, and the non-glyceride esters of the omega fatty acids, and mixtures thereof.

7. The formulation of claim 1, wherein the phospholipid comprising omega fatty acids is selected from the group consisting of α-linolenic acid (ALA), stearidonic acid, eicosatetraenoic acid, eicosapentaenoic acid (EPA), docosapentaenoic acid, docosahexaenoic acid (DHA), linoleic acid, gamma-linolenic acid, eicosadienoic acid, dihomo-gamma-linolenic acid, arachidonic acid, docosadienoic acid, adrenic acid, docosapentaenoic acid, oleic acid, eicosenoic acid, mead acid, erucic acid and nervonic acid, and combinations thereof.

8.-10. (canceled)

11. The formulation of claim 1, wherein the solubilizing agent is selected from the group consisting of Poloxamer 188, Polysorbate 80, Polysorbate 20, Vit E-TPGS, Solutol HS 15, PEG-40 Hydrogenated castor oil (Cremophor RH40), PEG-35 Castor oil (Cremophor EL), PEG-8-glyceryl capylate/caprate (Labrasol), PEG-32-glyceryl laurate (Gelucire 44/14), PEG-32-glyceryl palmitostearate (Gelucire 50/13); Polysorbate 85, Polyglyceryl-6-dioleate (Caprol MPGO), Mixtures of high and low HLB emulsifiers; Sorbitan monooleate (Span 80), Capmul MCM, Maisine 35-1, Glyceryl monooleate, Glyceryl monolinoleate, PEG-6-glyceryl oleate (Labrafil M 1944 CS), PEG-6-glyceryl linoleate (Labrafil M 2125 CS), Oleic acid, Linoleic acid, Propylene glycol monocaprylate (e.g. Capmul PG-8 or Capryol 90), Propylene glycol monolaurate (e.g., Capmul PG-12 or Lauroglycol 90), Polyglyceryl-3 dioleate (Plurol Oleique CC497), Polyglyceryl-3 diisostearate (Plurol Diisostearique) and Lecithin with and without bile salts, or combinations thereof.

12. The formulation of claim 1, wherein the water-soluble and lipophilic reducing agent are selected from the group consisting of L-ascorbic acid-6-palmitate, vitamin C and its salts alpha, beta, gamma and delta tocopherol or mixtures of tocopherol, and alpha, beta, gamma and delta-tocotrienols or mixtures thereof.

13. The formulation of claim 1, wherein the metal chelator is selected from the group consisting of ethylenediaminetetraacetic acid (EDTA), disodium EDTA and calcium disodium EDTA and mixtures thereof.

14. The formulation of claim 1, wherein the bisulfite is sodium bisulfite, potassium bisulfite, sodium metabisulfite or potassium metabisulfite.

15. The formulation of claim 1, wherein the formulation, when dissolved in water, provides a solution that remains stable toward degradation when stored at or below room temperature for a period of at least 6 months.

16. The formulation of claim 1, wherein the ratio of the solubilizing agent to the phospholipid comprising fatty acid ranges from about to 2:1 to 0.01:1.

17.-22. (canceled)

23. A stabilized aqueous emulsion of a phospholipid comprising fatty acid comprising:
a) phospholipid comprising omega fatty acid;
b) optionally, one or more solubilizing agent selected from the group consisting of solubilizing agents selected from the group consisting of Solutol HS 15, Cremophor EL, TPGS (polyoxyethanyl-a-tocopheryl succinate) or TPGS-1000 (D-alpha-tocopheryl polyethylene glycol 1000 succinate), wherein the tocopheryl is the natural tocopherol isomer or the un-natural tocopherol isomer, Poloxamer 188, Polysorbate 80, Polysorbate 20, Vit E-TPGS, Solutol HS 15, PEG-40 Hydrogenated castor oil (Cremophor RH40), PEG-35 Castor oil (Cremophor EL), PEG-8-glyceryl capylate/caprate (Labrasol), PEG-32-glyceryl laurate (Gelucire 44/14), PEG-32-glyceryl palmitostearate (Gelucire 50/13); Polysorbate 85, Polyglyceryl-6-dioleate (Caprol MPGO), Mixtures of high and low HLB emulsifiers; Sorbitan monooleate (Span 80), Capmul MCM, Maisine 35-1, Glyceryl monooleate, Glyceryl monolinoleate, PEG-6-glyceryl oleate (Labrafil M 1944 CS), PEG-6-glyceryl linoleate (Labrafil M 2125 CS), Oleic acid, Linoleic acid, Propylene glycol monocaprylate (e.g. Capmul PG-8 or Capryol 90), Propylene glycol monolaurate (e.g., Capmul PG-12 or Lauroglycol 90), Polyglyceryl-3 dioleate (Plurol Oleique CC497), Polyglyceryl-3 diisostearate (Plurol Diisostearique) and Lecithin with and without bile salts, or combinations thereof;
c) one or more additives selected from the group consisting of a metal chelator, a water soluble reducing agent, a lipophilic reducing agent, a bisulfite salt, a metabisulfite salt or mixtures thereof;
d) a carrier or additive selected from the group consisting of HI-CAP 100 (National Starch), Emcap Starch, TICAMULSION FC (TIC GUMS), Spray gum F (gum acacia with Maltrin-100), natural vanillin, natural maltol, maltodextrin 10-DE and mixtures thereof;
e) calcium disodium EDTA or disodium EDTA;
f) sodium bisulfite, sodium metabisulfite, potassium bisulfite or potassium metabisulfite; and
g) water, wherein the emulsion remains stable toward degradation when stored at or below room temperature for a period of at least 6 months.

24. (canceled)

25. The stabilized aqueous emulsion of claim 23, wherein the emulsion, when dissolved in water, the solution remains stable toward degradation when stored at or below room temperature for a period of at least 6 months.

26. A stabilized powder composition of a phospholipid comprising omega fatty acid comprising:
a) a phospholipid comprising omega fatty acid;
b) TPGS (polyoxyethanyl-a-tocopheryl succinate), Solutol HS 15 or Cremophor EL, or mixtures thereof;
c) a carrier or additive selected from the group consisting of HI-CAP 100 (National Starch), Emcap Starch, TICAMULSION FC (TIC GUMS), Spray gum F (gum acacia with Maltrin-100), natural vanillin, natural maltol, maltodextrin 10-DE and mixtures thereof;
d) calcium disodium EDTA or disodium EDTA; and
e) sodium bisulfite, potassium bisulfite, sodium metabisulfite or potassium metabisulfite; wherein the solution remains stable toward degradation when stored at or below room temperature for a period of at least 6 months.

27. A stabilized food, beverage, pharmaceutical or nutraceutical product comprising the stabilized powder composition of claim 26.
